(19) United States
(12) Patent Application Publication
(10) Pub. No.: US 2003/0134320 A1
(43) Pub. Date: Jul. 17, 2003

(54) Method system and computer program product for quality assurance in detecting biochemical markers

(75) Inventors: Jeffrey K. Barrus, Heber City, UT (US); John Henry Ryan, Salt Lake City, UT (US)

Correspondence Address: MYRIAD GENETICS INC., LEGAL DEPARTMENT, 320 WAKARA WAY, SALT LAKE CITY, UT, 84108, US

(73) Assignee: Myriad Genetics, Incorporated, Salt Lake City, UT

(21) Appl. No.: 10/345,905

(22) Filed: Jan. 15, 2003

Related U.S. Application Data
(60) Provisional application No. 60/349,165, filed on Jan. 15, 2002.

(51) Int. Cl. C12Q 1/68; G06F 19/00; G01N 33/48; G01N 33/50

(52) U.S. Cl. 435/6; 702/20

(57) ABSTRACT

A method is disclosed for providing a certified biochemical profile of a biological sample. The biochemical profile includes a plurality of data objects for a plurality of molecular markers. The method comprises: (1) providing a first data set for the plurality of molecular markers of the biological sample by a first process from a first electrical signal representing a first unprocessed image data; (2) providing a second data set for said plurality of molecular markers of the biological sample by a second process from a second electrical signal representing a second unprocessed image data, wherein the first process is different from said second process; and (3) comparing, by a computer-readable program code, the first and second data sets, whereby a certified biochemical profile is generated if no discrepancy between the first and second data sets are detected. Preferably, the steps of the method are coordinated by another computer-readable program code. Systems and computer program products embodying the method or useful in the method are also disclosed.

### RELATED U.S. APPLICATION

This application claims priority to U.S. Provisional Application Serial No. 60/349,165, filed on, Jan. 15, 2002, the content of which is incorporated herein by reference in its entirety.

### FIELD OF THE INVENTION

The present invention generally relates to bioinformatics, and particularly to methods, systems, and computer program products for providing a profile of biochemical markers of a biological sample and performing quality assurance on the profile.

### BACKGROUND OF THE INVENTION

With the advance of research equipments and technologies, biochemical experiments are becoming increasingly high throughput and efficient. For example, microarrays are more and more frequently used in genomic and proteomic research to allow simultaneous detection of tens of thousands molecular markers such as mRNAs or proteins. New DNA sequencing technologies are also being developed, which are expected to increase DNA sequencing speed exponentially. Typically, such biochemical experiments involve labeled molecules, which give rise to detectable signals. Such signals are detected and converted to data sets representing the physical attributes of the detected molecules. This data acquisition and analysis process is complicated by two factors: irregularity inherent in the experiments and the large amount of data typically involved.

Take the process of STR (short tandem repeat) profiling as an example, specific primer sets uniquely suited to the desirable STR loci are used in PCR reactions to amplify the STR loci and produce a plurality of fluorescence-labeled DNA fragments. The number of repeats in a STR locus is deduced from the size of the amplified DNA fragment corresponding to that locus. Thus, when separated by gel electrophoresis, fluorescence signals from the labeled DNA fragments are acquired and converted into an electrical signal, which is then processed using a suitable algorithm to make genotype calls, i.e., to determine the number of repeats in each labeled DNA fragment. However, size differences among different alleles can be as small as a few base pairs. Also, secondary “shadow” bands or “snowdrifts” often show up in gel images along with the primary bands corresponding to the amplified DNA fragments. In addition, the mobility of DNA fragments can be unstable or inconsistent even within a single sequencing gel. All these and other problems often complicate the data analysis and genotype calling process, making the results unreliable.

Various software programs have been designed to speed up the data analysis and genotype calling process. For example, U.S. Pat. Nos. 5,541,067; 5,580,728; 5,876,933; and 6,054,268 all entitled “method and system for genotyping” disclose such a method. The method includes the steps of converting labels of DNA fragments amplified from one or more locations into a first electrical signal, removing a reproducible pattern of the amplification from the first electrical signal to form a third electrical signal, and producing from the third electrical signal a genotype at the locations. The method is purported to be amenable to full automation for genotype calling with increased accuracy. However, the method is based on the refinement of the first electrical signal and relies on one single analysis system. As a result, a strong bias can be introduced in the data analysis and genotype calling process.

Therefore, with an ever-increasing amount experimental data being produced from biochemical experiments using labeled nucleic acids, proteins, or cells, there is continued need for methods of processing such data with increased accuracy and reliability.

### SUMMARY OF THE INVENTION

A computer-implemented method is disclosed for providing a certified biochemical profile (e.g., STR profile, nucleic acid hybridization or protein binding profile) of a biological sample. The method employs two independent analysis paradigms and compares the results for purposes of quality assurance. The method produces a quality assurance report and certifies the results in the absence of a discrepancy between the results of the two independent analysis paradigms. The method can be implemented in a fully automated manner. Thus, the method eliminates the strong bias typically resulting from the traditional single analysis paradigm without sacrificing the efficiency of the analysis process.

In general, the method includes the steps of (1) providing, from a first electrical signal corresponding to a first unprocessed image data and by a first process, a first data, set representing the plurality of molecular markers of the biological sample; (2) providing, from a second electrical signal corresponding to a second unprocessed image data and by a second process, a second data set representing the plurality of biological markers of the biological sample, wherein the first process is different from the second process; and (3) comparing, by a computer-readable program code, the first and second data sets to determine the presence or absence of a discrepancy between the first and second data sets, whereby a certified biochemical profile is generated if no discrepancy between the first and second data sets is detected. Preferably, the providing steps and the comparing step of the method are coordinated by a computer-readable program code such that the comparing step is automatically performed when the first and second data sets are both provided in comparable formats.

In one embodiment, the method is used in providing a DNA STR profile of an individual, and comprises the steps of: (1) producing a plurality of labeled DNA fragments having detectable labels and corresponding to the DNA STR profile of the individual; (2) producing an electrical signal representing unprocessed image data of the labeled DNA fragments (3) providing a first data set representative of the number of short tandem repeats in each of the plurality of labeled DNA fragments, by a first process from the electrical signal, the first process comprising the steps of normalizing the electrical signal, and converting the electrical signal to the first data set; (4) providing a second data set representative of the number of short tandem repeats in each of the plurality of labeled DNA fragments, by a second process from the electrical signal, the second process comprising converting the electrical signal to the second data set, wherein the second process is different from the first process; and (5) comparing, as enabled by a computer program code, the first data set with the second data set to determine the presence or absence of a discrepancy between the first and second data sets and to provide a QA report, wherein a certified STR profile is provided comprising the first or second data set in the absence of the discrepancy. Preferably, the providing steps and the comparing step are coordinated by a computer-readable program code such that the comparing step is automatically performed when the first plurality of data objects and the second plurality of data objects are both provided in comparable formats.

In another embodiment, the method is applied to the analysis of an image data obtained from a microarray having proteins, nucleic acids, tissues or cells in a plurality of loci of the microarray. The method comprises: (1) providing an electrical signal representing unprocessed image data from the microarray; (2) producing a first data set representative of the identity of the plurality of loci and image intensity at the plurality of loci, by a first process from the electrical signal, the first process comprising the steps of normalizing the electrical signal, and converting the electrical signal to the first data set; (3) producing, from the electrical signal and by a second process, a second data set representative of the identity of the plurality of loci and image intensity at the plurality of loci, the second process comprising converting the electrical signal to the second data set, wherein the second process is different from the first process; and (4) comparing, by a computer-readable program code, the first data set with the second data set to determine the presence or absence of a discrepancy between the first and second data sets, wherein the producing steps and comparing step are coordinated by a computer-readable program code such that the comparing step is automatically performed when the first data set and the second data set are both provided in a comparable format.

In another aspect, systems are provided for implementing the methods of the present invention. The system comprises a processor capable of effecting the following steps within the system: (1) providing, from an electrical signal corresponding to an unprocessed image data and by a first process, a first data set corresponding to the plurality of molecular markers of the biological sample, the first process comprising the steps of normalizing the electrical signal, and converting the normalized electrical signal to the first data set; (2) providing, from the electrical signal and by a second process, a second data set corresponding to the plurality of biological markers of the biological sample, the second process comprising the steps of normalizing the electrical signal and converting the normalized electrical signal to the second data set, wherein the first process is different from the second process; and (3) comparing, by a first computer-readable program code, the first and second data sets to determine the presence or absence of a discrepancy between the first and second data sets and to produce a QA report. Preferably, the providing steps and the comparing step are coordinated under instructions from a second computer-readable program code such that the comparing step is automatically performed when the first data set and the second data set are both provided in a comparable format.

In one embodiment of the system, the system is useful to DNA STR profiling and comprises: (1) a sample analyzer for separating a plurality of labeled DNA fragments; (2) a data collector for producing an electrical signal representing unprocessed image data of the plurality of labeled DNA fragments; (3) an interface module for receiving from the data collector the electrical signal; (4) a first computer program means for producing, from the electrical signal and by a first process, a first data set corresponding to the number of short tandem repeats in each of the plurality of labeled DNA fragments; (5) a second computer program means for producing, from the electrical signal and by a second process, a second data set corresponding to the number of short tandem repeats in each of the plurality of labeled DNA fragments, wherein the first and second computer program means are different from each other; and (6) a third computer program means for comparing the first and second data sets to determine the presence or absence of a discrepancy between the first and second data sets and to produce a QA report. Preferably, the processor is also capable of effecting the coordination of the providing steps and the comparing step.

In another embodiment, the system is suitable for profiling a microarray having proteins, nucleic acids, tissues or cells in a plurality of loci of the microarray, and comprises (1) a data collector for producing an electrical signal representing unprocessed image data of the microarray; (2) an interface module for receiving from the data collector the electrical signal; (3) a first computer program means for producing, from the electrical signal and by a first process, a first data set corresponding to the identity of the plurality of loci and image intensity at the plurality of loci; (4) a second computer program means for producing, from the electrical signal and by a second process, a second data set corresponding to corresponding to the identity of the plurality of loci and image intensity at the plurality of loci, wherein the first and second computer program means are different from each other; and (5) a third computer program means for comparing the first and second data sets to determine the presence or absence of a discrepancy between the first and second data sets and to produce a QA report. Preferably, the system further comprises a fourth computer program means interfacing with the first, second and third computer program means to coordinate the progress thereof.

In yet another aspect of the present invention, a computer program product is provided for providing a certified biochemical profile of a biological sample, which includes a data set having a plurality of data objects representing a plurality of molecular markers. The computer program product comprises a computer-usable medium having computer-readable program code embodied thereon for effecting the following steps within a computing system: (1) receiving a first data set corresponding to the plurality of molecular markers of the biological sample, wherein the first data set is converted from an electrical signal corresponding to an unprocessed image data by a first process, the first process comprising the steps of normalizing the electrical signal, and converting the normalized electrical signal to the first data set; (2) receiving a second data set corresponding to the plurality of molecular markers of the biological sample, wherein the second data set is converted from an electrical signal corresponding to an unprocessed image data by a second process, the second process comprising the steps of normalizing the electrical signal and converting the normalized electrical signal to the first data set, wherein the first process is different from the second process; (3) comparing the first and second data sets to determine the presence or absence of a discrepancy between the first and second data sets and to produce a QA report; and (4) coordinating the first and second processes such that the comparing step is performed when the first data set and the second data set are both provided in a comparable format.

The foregoing and other advantages and features of the invention, and the manner in which the same are accomplished, will become more readily apparent upon consideration of the following detailed description of the invention taken in conjunction with the accompanying drawings and examples, which illustrate preferred and exemplary embodiments.

### BRIEF DESCRIPTION OF THE DRAWINGS

FIG. 1A is a flowchart illustrating an embodiment of the method of the present invention;

FIG. 1B is a flowchart illustrating another embodiment of the method of the present invention;

FIG. 1C is a flowchart illustrating another embodiment of the method of the present invention;

FIG. 2 is a flowchart showing an exemplary algorithm for comparing two different data sets in the method of the present invention;

FIG. 3 illustrates an exemplary algorithm for coordinating the data analysis processes and data comparison;

FIG. 4 is a flowchart demonstrating the coordinated steps of data analysis and comparison;

FIG. 5 illustrates an embodiment in which the method of the present invention is applied to STR profiling;

FIG. 6 is a flowchart showing the steps involved in a data analysis process using the ABI Genotyper®;

FIG. 7 is a diagram of sized data in STR profiling using GeneScan with MacIntosh™ system macro (KeyQuencer™);

FIG. 8 is a flowchart illustrating an embodiment of the present invention as applied to STR profiling;

FIG. 9 is a flowchart showing an exemplary process according to the present invention involving computerized coordination;

FIG. 10 is a flowchart illustrating another embodiment of the present invention as applied to STR profiling;

FIG. 11 shows another embodiment of the present invention as applied to STR profiling;

FIG. 12 is another flowchart illustrating an embodiment of the present invention as applied to STR profiling, in which the entire process is fully automated;

FIG. 13 illustrates examples of data sets representing STR profiles and an example of a QA report;

FIG. 14 illustrates an embodiment of the present invention as applied to a microarray-based analysis;

FIG. 15A shows an embodiment of the system for providing a validated biochemical profile according to the present invention;

FIG. 15B illustrates another embodiment of the system for providing a validated biochemical profile according to the present invention;

FIG. 16 is a schematic diagram illustrating a computing system for executing the instructions from the computer program products or computer-readable codes of the present invention.

### DETAILED DESCRIPTION OF THE INVENTION

In accordance with a first aspect, the present invention relates to methods for providing a certified biochemical profile of a biological sample having a data set including a plurality of data objects representing a plurality of molecular markers. The methods include the steps of (1) providing, from a first electrical signal corresponding to a first unprocessed image data and by a first process, a first data set representing the plurality of molecular markers of the biological sample; (2) providing, from a second electrical signal corresponding to a second unprocessed image data and by a second process, a second data set representing the plurality of biological markers of the biological sample, wherein the first process is different from the second process; and (3) comparing, under instructions of a computer-readable program code, the first and second data sets to detect discrepancies between the first and second data sets, whereby a certified biochemical profile is generated if no sufficient number of discrepancies between the first and second data sets is detected. It is noted that the stringency of the requirements for certifying the biochemical profile can vary. That is, the allowable number of discrepancies between the two data sets for the computer to certify the results can be preset by users. Preferably, the providing steps and the comparing step of the method are coordinated by a computer-readable program code such that the comparing step is automatically performed when the first and second data sets are both provided in comparable formats.

In the methods, the first unprocessed image data can be the same as, or different from the second unprocessed image data. However, in any case, they are obtained from the same biological sample.

The method can be applicable to a variety of biochemical profiling involving various molecular markers. For example, the method can be used in obtaining genotype profiles for any cells or biological organisms. In particular, the method is useful in DNA sequencing, SNP profiling and microsatellite analysis such as short tandem repeats (STR) profiling in humans and other organisms. As used herein, the term “genotype profile” means a collection of information derived from DNA obtained from a subject of interest that is useful as markers characterizing that particular subject. Examples of “DNA profile” include SNP profiles, which include one or more single nucleotide polymorphic markers (SNPs), and STR profiles, which include one or more data reflecting the number of particular short tandem repeats at one or more pre-determined chromosome loci.

The method is also useful in protein analysis applications, such as one-dimensional protein gel or two-dimensional protein gel analysis. In these applications, typically, proteins are labeled with detectable labels and separated by gel electrophoresis or other techniques. Signals or image caused by the labels associated with the separated protein bands or spots can be detected and acquired by a sensing device which produces an electrical signal corresponding to the signals or image. The electrical signal can be processed using the method of the present invention to derive data sets representing the sizes, pIs and/or concentrations of the proteins associated with the protein bands or spots. Such data sets represent the attributes of the protein binds or spots (i.e., biochemical markers) which form a protein profile.

The method is also applicable to processing images acquired from microarrays including nucleic acid arrays, protein arrays, tissue arrays as well as cell arrays. Typically, a microarray with nucleic acids hybridized thereto or proteins bound thereon emits detectable signals from a plurality of arrayed spots on the microarray. Each spot includes a molecular marker such as a particular protein or mRNA. An array profile would include the location/identity of the spots (i.e., molecular markers) and the signal intensity associated with the spots (i.e., molecular markers).

In addition, as will be apparent from the description below, the method of the present invention would also be useful in analyzing other multi-dimensional data such as histogram data, flow cytometry data, etc.

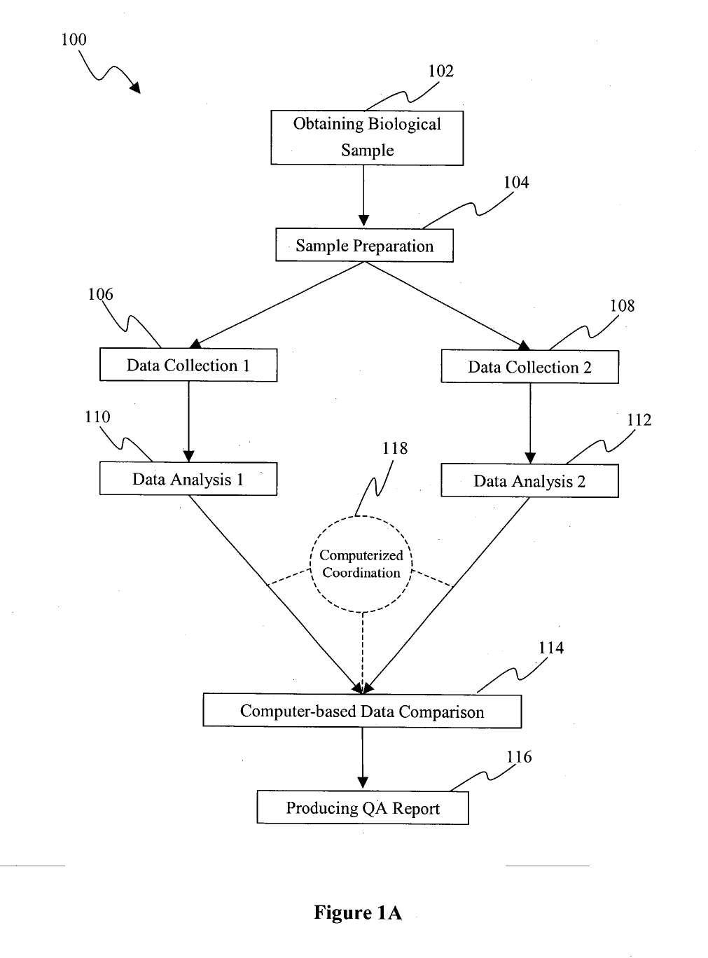

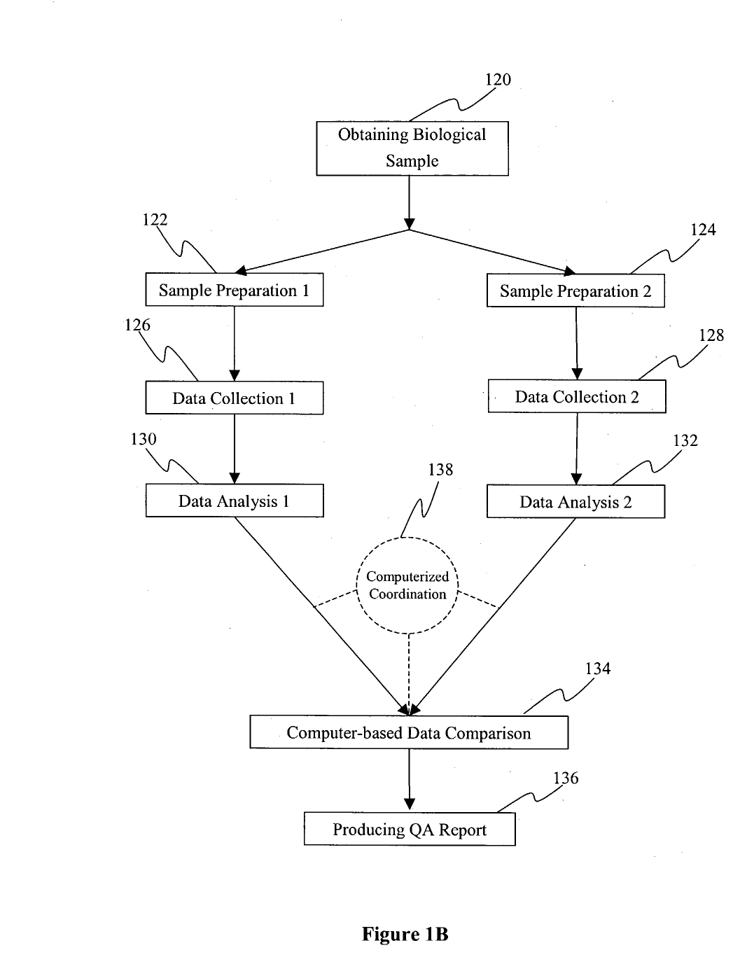

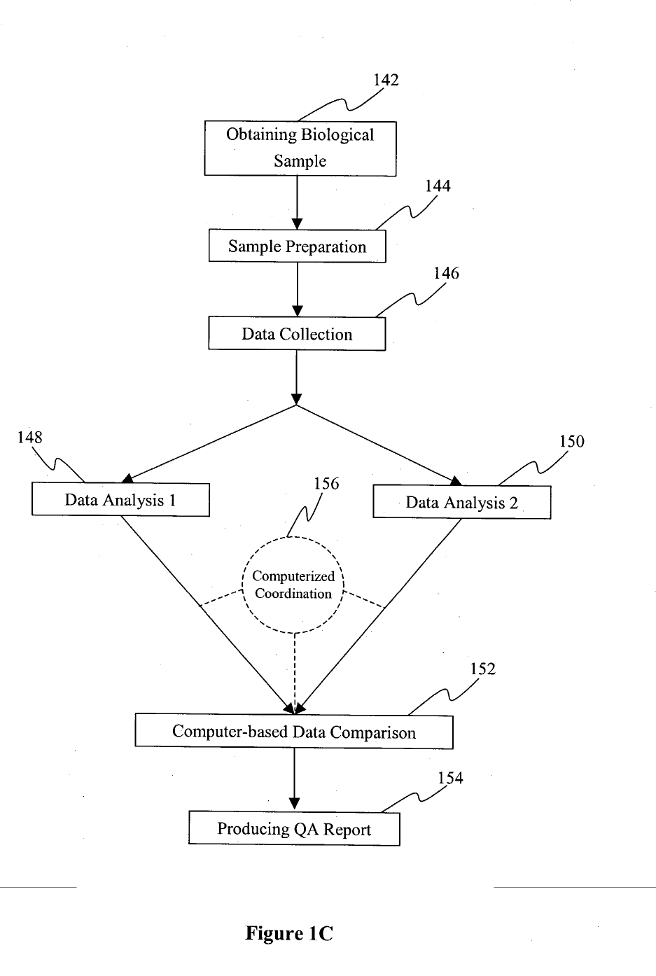

The method of the present invention may take a variety of forms regardless of the type of a biochemical profile. For example, in one embodiment as shown in FIG. 1A, the method 100 includes step 102 to obtain a biological sample. To provide a genotype profile, the biological sample can be a DNA or tissue or cell sample containing a sufficient amount of DNA material. For purposes of providing a gene expression profile on a microarray, the biological sample should contain mRNAs or cDNAs representative of the sample. Likewise, to provide a protein profile or protein microarray profile, the biological sample should include proteins, or tissues or cells containing a sufficient amount of proteins representative of the sample.

Once a biological sample is obtained, the sample can be processed in step 104 so that multi-dimensional unprocessed image data can be obtained from the processed sample. For example, in an STR analysis, a biological sample containing genomic DNA can be used in multiplex PCR amplification using suitable primer sets to amplify selected STR loci in the presence of one or more fluorescent markers. Different colors may be used to labeled different primer sets in a multiplex PCR reaction for DNA amplification. The amplified, fluorescence-labeled DNA fragments can then be subject to gel electrophoresis or capillary sequencing to separate the DNA fragments. In the case of gene expression profiling, fluorescence-labeled mRNA or cDNA prepared from a biological sample can be hybridized to an oligonucleotide array.

The light signals from the fluorescent label can be collected using two different data collection means (or data collectors) in steps in 106 and 108, respectively. For example, two different image scanning hardware and/or software may be used in steps 106 and 108, respectively, to produce a first and second electrical signal, respectively, representing unprocessed image data of the fluorescence-labeled DNA fragments in STR profiling or the fluorescence-labeled mRNA or cDNA associated with different array spots in gene expression profiling. In step 110, the electrical signal produced in step 106 is analyzed using a first data analysis algorithm to provide a set of STR genotype data (in STR profiling) or gene expression data (in gene expression profiling). In step 112, the electrical signal produced in step 108 is analyzed using a second data analysis algorithm to provide a second set of STR genotype data (in STR profiling) or gene expression data (in gene expression profiling). The first data analysis algorithm is different from the second analysis algorithm. However, the results generated by steps 110 and 112 should be in formats so that they can be compared, or the results can be converted into such forms. Step 114 is a comparing step in which the first data set is compared to the second data set using a computer or an equivalent automatic device thereof driven by computer software. Optionally, in step 114, the first and/or second data sets are formatted so that they are comparable in step 114. The computer-based comparison in step 114 compares each data in the first data set to its corresponding data in the second data set, and identifies the mismatches. That is, the computer identifies those data in the first and second data sets that are not identical to each other, and includes the mismatches in a quality assurance (QA) report produced in step 116. When all data in one set match their corresponding data in the other set, the QA report indicates so and certifies the profile to be the accurate profile. When one or more mismatches are identified in the QA report, a quality assurance specialist may become involved to review the collected raw data and analyze genotype data in an attempt to resolve the mismatches. If the mismatches are still not explainable, then the sample may be reprocessed and re-analyzed. The QA report may also certify the results when there are one or more mismatches. As will be apparent to skilled artisans, the stringency of the certification can be predetermined at any level. In addition, the QA report may also indicate the quality of the processed data and consistency.

In preferred embodiments, computer-based coordination (step 118) is provided so that the computer automatically performs step 114 once the results from steps 110 and 112 are both available in comparable formats and ready for comparison.

FIG. 1B illustrates another embodiment of the method of the present invention. In this embodiment, the biological sample obtained in step 120 is divided and processed using two different sample preparation or processing approaches as shown in steps 122 and 124, respectively, so that multi-dimensional unprocessed image data can be obtained from the processed sample. The image data can be collected using two different data collection means (or data collectors) in steps 126 and 128, respectively. Each collected data in the form of an electrical signal is analyzed in steps 130 and 132. The data collection techniques used in steps 126 and 128 may be the same or different, preferably different. Likewise, the data analysis means in steps 130 and 132 may be the same or different, preferably different. The data sets generated in steps 130 and 132 are compared in step 134 using a computer to generate a QA report in step 136. The QA report either certifies the biochemical profile or identifies the mismatches in the two data sets. Preferably, computer-based coordination (step 138) is provided so that a computer automatically performs step 134 when the results from steps 130 and 132 are both available in comparable formats and are ready for comparison.

A preferred embodiment of the method of the present invention is summarized in FIG. 1C. As shown in the figure, two independent data analysis steps (148 and 150) are performed on the same data collected (in step 146) (in the form of an electrical signal representing unprocessed image data) from the same processed sample (step 144) based on the biological sample obtained in step 142. Two distinct data analysis algorithms are employed in the two independent data analysis steps. The two data sets generated in steps 148 and 150 are compared by computer under the instructions of a computer-readable program code in step 152, and a QA report is produced in 154 based on the computer-based comparison. Preferably, computer-based coordination (step 156) is provided so that a computer automatically performs step 152 when the results from steps 148 and 150 are both available in comparable formats and are ready for comparison. The coordination is performed by a computer program means, which interfaces with both the data analysis means and the comparison means.

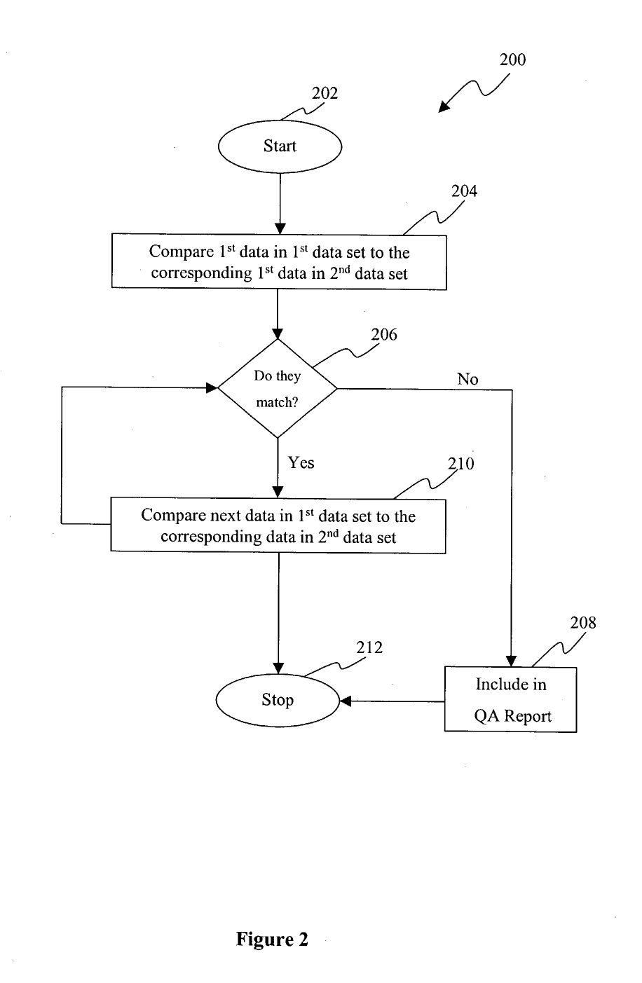

FIG. 2 is a flowchart illustrating an embodiment of the operation and control flow 200 in the computer-based comparison step of the method of the present invention. It is noted that the embodiment in FIG. 2 is for exemplary purposes only and is not intended to limit the scope of the present invention. As shown in FIG. 2, control flow 200 begins with the step 202 and proceeds to step 204 in which the first data in the first data set is compared to the corresponding first data in the second data set. In step 206, the result of the comparison is determined. If the two data match each other, the computer proceeds to compare (step 210) the next data in the first data set to the corresponding next data in the second data set. Otherwise, the computer includes the non-matching data in the QA report in step 208. The procedure is repeated until all data in the two data sets have been compared, and then the control flow stops at step 212. Optionally, before the step 204, the control flow 200 also includes a formatting step, in which the two data sets to be compared are formatted such that they can be compared in step 204.

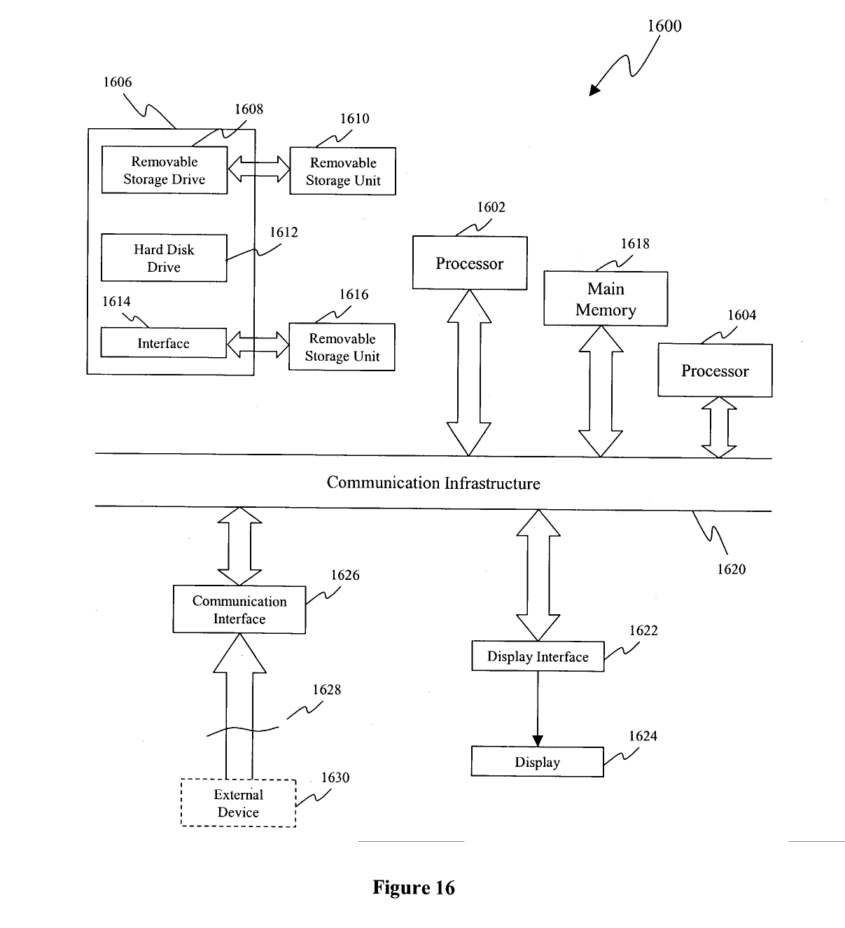

In the method of the present invention, control flow 200 is implemented using hardware, software or a combination thereof in one or more computer systems or other processing systems (e.g., the computer system 1600 shown FIG. 16). The control flow 200 in the present invention can be implemented in any suitable language and/or browsers. For example, control flow 200 may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment.

In another example, the control flow 200 can be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™—or JavaScript™—enabled browsers such as HotJava™, Microsoft™ Explorer™, or Netscape™ can be used. When active content web pages are used, they may include Java™ applets or ActiveX™ controls or other active content technologies.

As discussed above, in preferred embodiments of the methods of the present invention, the two different data analysis processes and the comparison step are coordinated by a computer means (e.g., step 118, 138, and 156 in FIGS. 1A, 1B and 1C, respectively). In such preferred embodiments, the entire process can be automated and human intervention can be obviated. The efficiency of the process is greatly increased. In one embodiment, the computer-based coordination involves monitoring the availability of both results of the two different data analysis processes, determining whether the results are in comparable formats, and causing the initiation of the comparison of the two results.

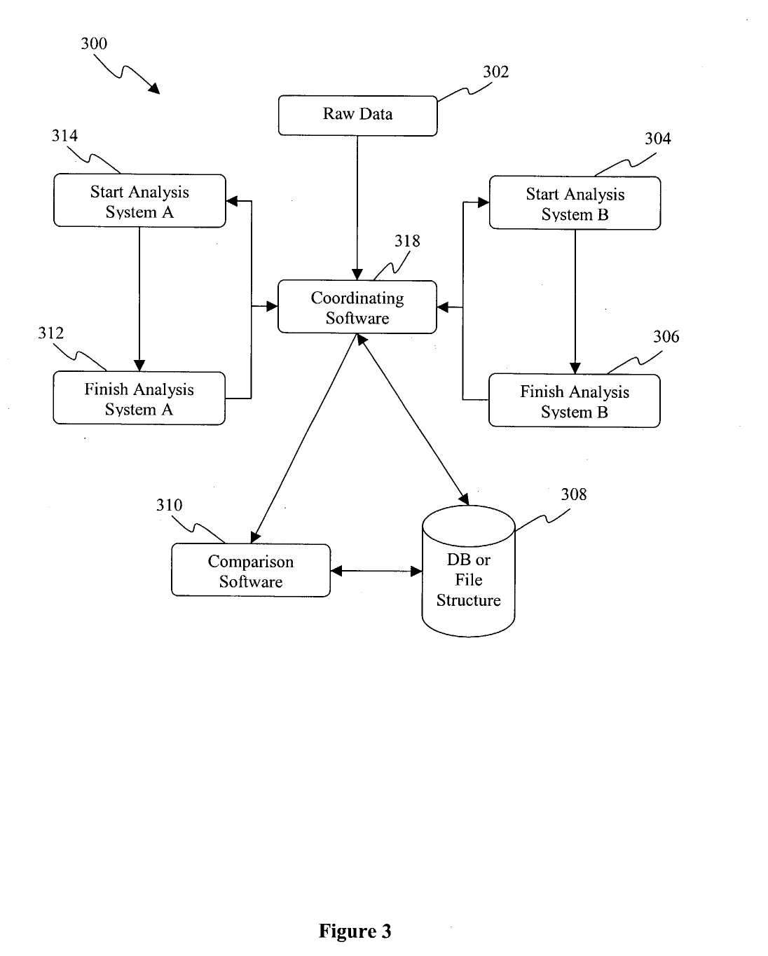

FIG. 3 is a flowchart illustrating an embodiment of the operation and control flow 300 for implementing the computer-based coordination function in the method of the present invention. It is noted that the embodiment in FIG. 3 is for exemplary purposes only and is not intended to limit the scope of the present invention. As shown in FIG. 3, the computer-based coordination means, e.g., enabled by a computer-readable program code or software interacts with the two different data analysis systems and monitors the start and completion of the analysis. In addition, the coordination means also interacts with the computer-based comparison means as well as a database or file structure which stores the data sets from the analysis processes and/or the comparison results.

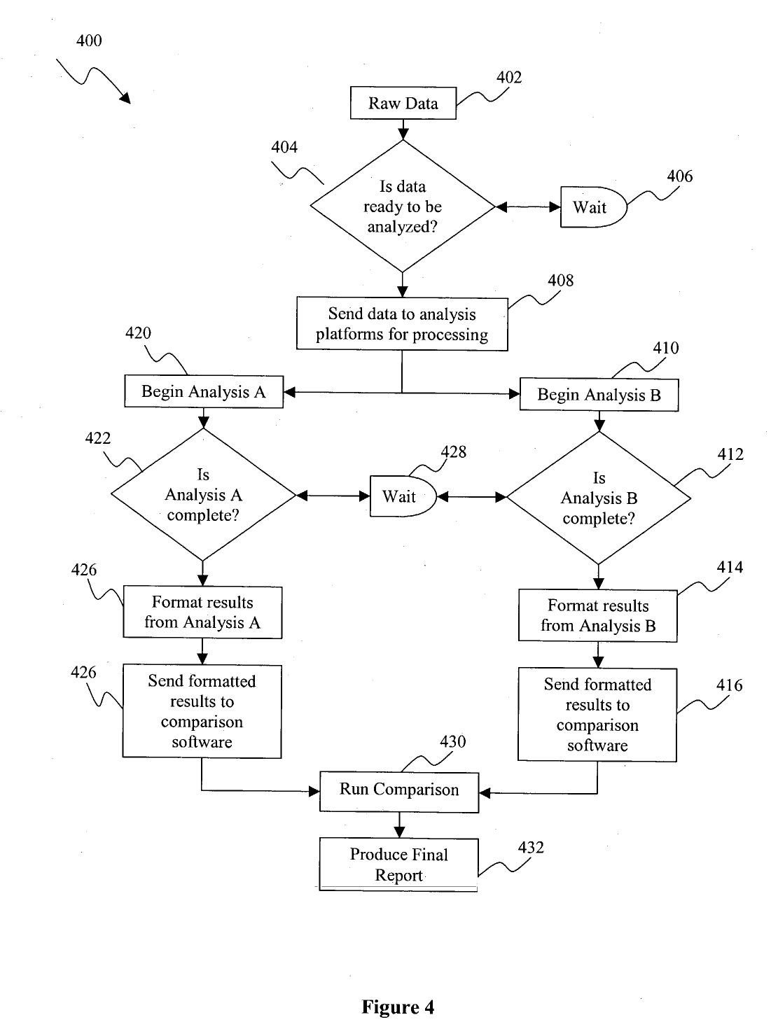

FIG. 4 is another flowchart illustrating an embodiment of the method of the present invention in which certain steps are closely monitored and coordinated by computer-based programs. Referring to process 400 in FIG. 4, a first coordination performed in steps 404 and 406 ensures that the unprocessed image data provided in step 402 is ready for processing. When ready, the unprocessed image data is sent (step 408) to two different data analysis platforms for processing. That is, the electrical signal representing the unprocessed image data is processed separately by Analysis B (steps 410 and 412) and Analysis A (steps 420 and 422). A computer program is employed to coordinate the two different analysis processes. As shown in steps 422, 412 and 428, the computer program determines whether the analysis processes are completed and controls the initiation of the following steps. That is, when both analysis processes are completed and data sets are available, the coordination means signals the initiation of the formatting of the data sets (steps 414 and 424), which are then processed by the computer program for data comparison (step 430) to produce the final QA report (step 432).

The computer-based coordination function (e.g., those illustrated in FIGS. 3 and 4) in the methods of the present invention can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the coordination means can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™—or JavaScript™—enabled browsers such as HotJava™, Microsoft™ Explorer™, or Netscape™ can be used. When active content web pages are used, they may include Java™ applets or ActiveX™ controls or other active content technologies.

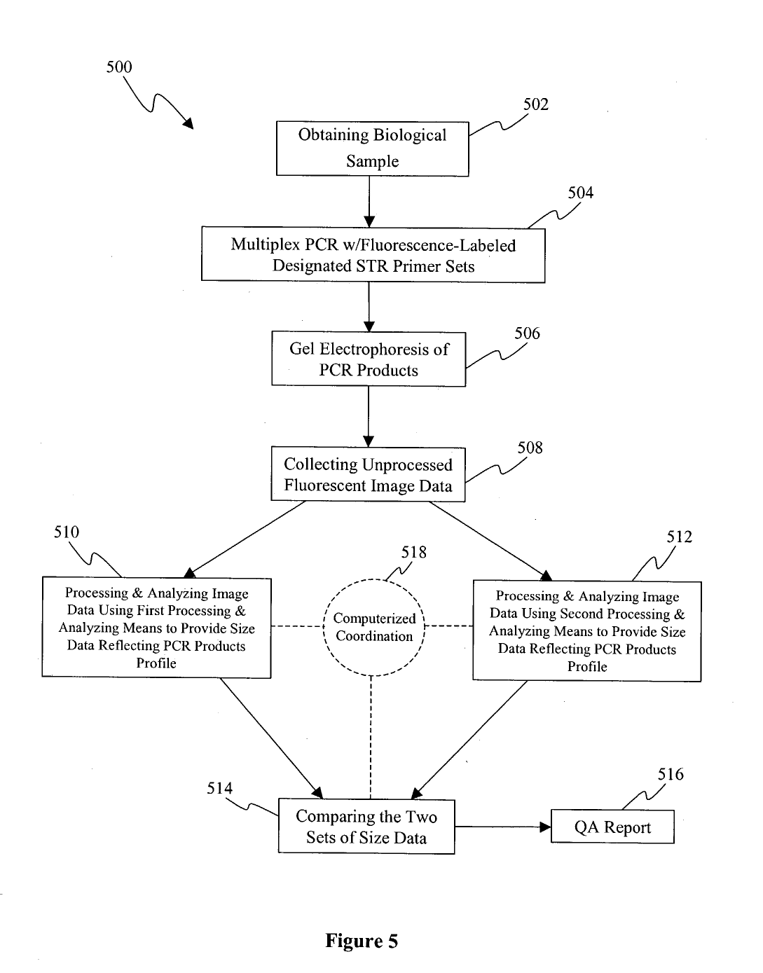

FIG. 5 is a flowchart depicting an embodiment of the method of the present invention as applied to STR profiling. Typically, STR profiling includes detecting STR markers in a number of chromosome loci. For example, the Combined DNA Index System (CODIS) database requires STR information at 13 core tetranucleotide STR loci including CSF1PO, D3S1358, D5S818, D7S820, D8S1179, D13S317, D16S539, D18S51, D21S11, vWA, FGA, TH01, and TPOX. Additionally, the Amelogenin locus is typically determined for gender identification. Therefore, the biological sample obtained from an individual to be STR profiled in step 502 should include DNA materials encompassing one or more of the forgoing loci. Any tissue or cell sample containing such DNA materials may be useful. For this purpose, a tissue sample containing cell. nucleus and thus genome DNA can be obtained from the individual to be profiled. Blood samples can also be useful except that only white blood cells and other lymphocytes have cell nucleus, while red blood cells are a nucleus and contain mRNA.

In step 504, the biological sample is processed by PCR amplification. The tissue or cell samples can be included directly in PCR reactions without much processing. Alternatively, genomic DNA can be extracted and/or purified. For purposes of PCR amplification of the above loci required by CODIS, various commercial available PCR amplification kits may be used, including, e.g., the AmpFLSTR® Profiler Plus™ PCR Amplification Kit, the AmpFLSTR® COfiler™ PCR Amplification Kit, and the AmpFLSTR® Identifiler™ PCR Amplification Kit, all available from Applied Biosystems, Inc., Foster City, Calif. The kits generally include fluorescence markers, polymerase, and specific primer sets uniquely suited for the desirable STR loci to be amplified.

As a result of the PCR amplification, a plurality of DNA fragments are produced. The length of the DNA fragments reflects the numbers of the short tandem repeats in those loci in the individual's chromosomes. The PCR products comprising the plurality of amplified labeled DNA fragments are then subject to gel electrophoresis in step 506, or alternatively capillary sequencing. As will be apparent to skilled artisans apprised of the present invention, any sample analyzer for separating the plurality of labeled DNA fragments can be useful. Examples of automated sequencing and DNA fragment analysis instruments include the ABI PRISM® 310 Genetic Analyzer, the ABI PRISM® 3700 DNA Analyzer and the ABI PRISM® 377 DNA Sequencer all commercially available from Applied Biosystems, Inc., Foster City, Calif. Other similarly useful gel electrophoresis instruments include those commercially available from Amersham Pharmacia Bioscience, Inc. and Hitachi FMBIO® II from Hitachi Inc.

In step 508 of process 500, unprocessed fluorescent image data is collected from the gel after electrophoresis is performed in step 506. The unprocessed fluorescent image data is typically collected from the gel by a tracking device (e.g., laser scanning monitor) in conjunction with data collection software. The raw gel data, i.e., unprocessed fluorescent image data, is then processed and analyzed by two distinct means or algorithms in parallel as shown in steps 510 and 512 in FIG. 5. The steps 510 and 512 convert the electrical signal representing the unprocessed image data to two different sets of allele call data indicating the numbers of short tandem repeats in the STR loci amplified. Preferably, the two different data sets are formatted such that they can be compared in step 514. The computer-based data comparison in step 514 can be performed as described above, which provides a quality assurance report in step 516.

Preferably, a computer program means is provided to coordinate the two data processing steps 510 and 512, and the comparison step 514 such that the comparison step is automatically performed when the two data sets are both provided in comparable formats.

Any data processing and analyzing methods may be used in steps 510 and 512 so long as the methods in steps 510 and 512 are different from each other in one way or another. That is, the two methods in steps 510 and 512 are embodied in two different paradigms of algorithms. Preferably, the two methods use one or more different algorithms. For example, the methods typically employ a matrix to process raw gel data by normalizing the intensity of the fluorescent light and deciphering and eliminating spurious peaks (or so called “snowdrifts”). In addition, the methods also include steps of analyzing the shapes and heights of the light peaks and determining the identities of the alleles. The two methods employed in steps 510 and 512 can vary in any of such aspect(s). For example, in one embodiment, the method in step 510 may employ algorithms that conduct low threshold analysis (i.e., using low level filters) of the diagram reflecting the intensity of the fluorescence signals on the gel by labeling all anomalies including spurious peaks as real peaks, while the algorithms utilized in step 512 conduct highly intelligent analysis (i.e., high threshold filter) of the shapes, heights and peak ratios of the fluorescence signal peaks to distinguish real peaks from artifacts.

Various computer software suitable for this purpose is commercially available. For example, the ABI PRISM® Genotyper® Software (Applied Biosystems, Inc., Foster City, Calif.) can be used in combination with the GeneScan® Analysis Software (also available from Applied Biosystems, Inc., Foster City, Calif.) to process and analyze the raw gel data in step 510 of the embodiment shown FIG. 5, while another commercial software such as the Hitachi FMBIO® Analysis Software (available from Promega Inc., Madison, Wis.) or TrueAllele™ (commercially available from Cytogenetics, Co., Pittsburgh, Pa.) is used in step 512. As will be apparent to skilled artisans, any alternative combinations of the various software may be adopted.

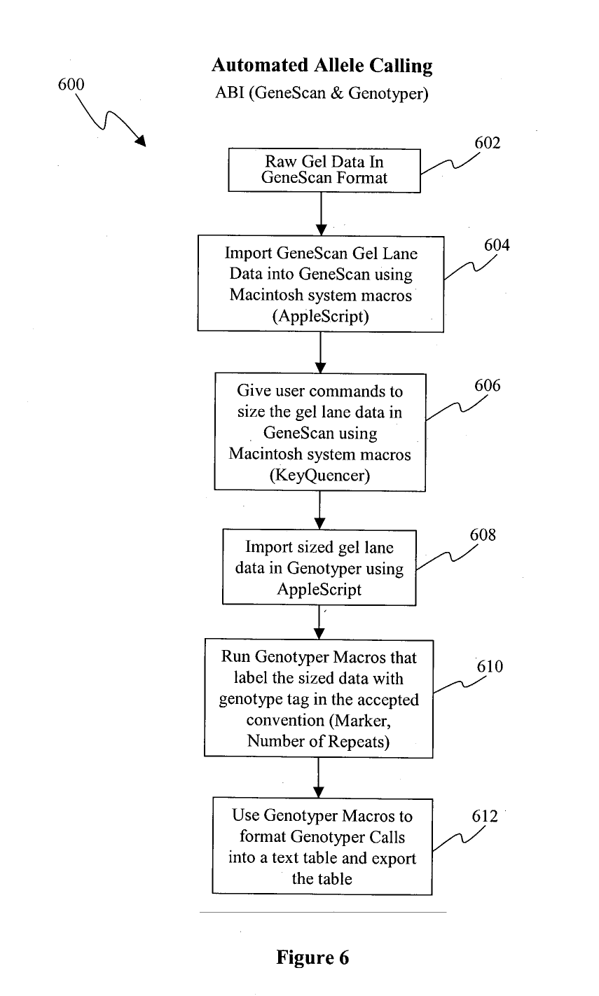

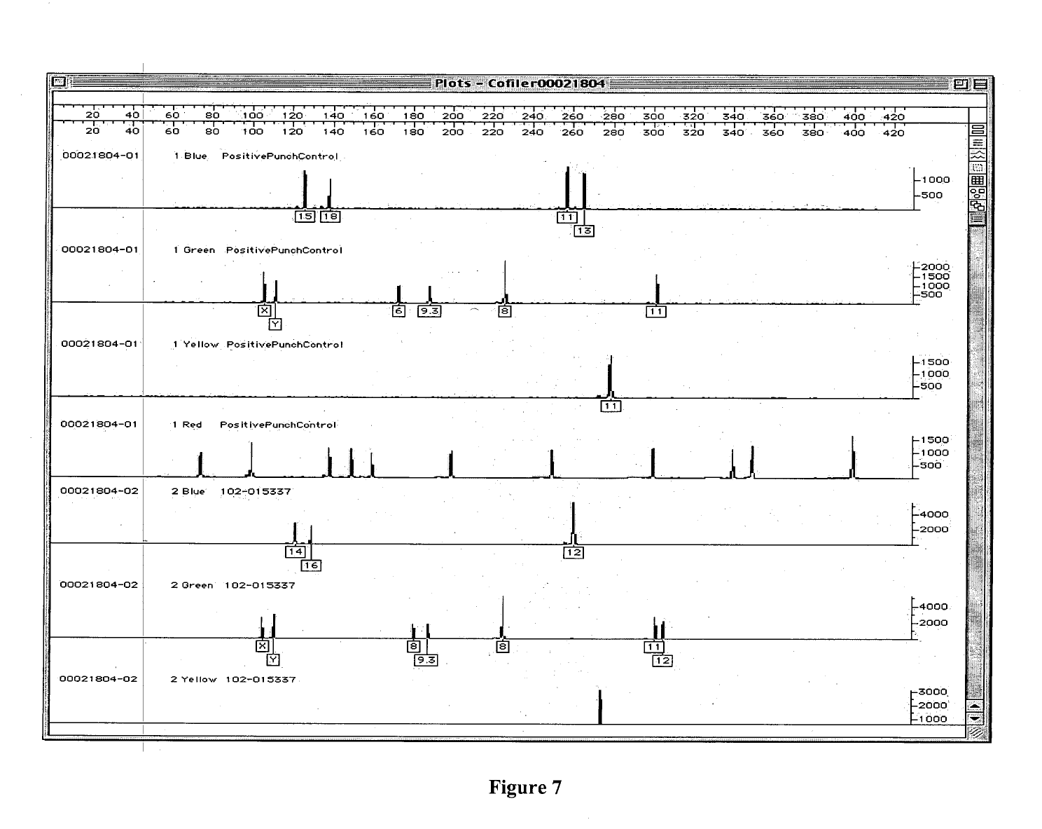

Referring to FIG. 6, the flowchart 600 illustrates further details in the data processing and analysis using the ABI PRISM® Genotyper® Software and the GeneScan® Analysis Software. The process begins with raw gel data in GeneScan Format in step 602. In step 604, the GeneScan Gel Lane Data is imported into GeneScan using MacIntosh system macros (AppleScript™). In step 606, the gel lane data is sized in GeneScan using Maclntosh™ system macro (KeyQuencer™). FIG. 7 shows a diagram of sized data generated in step 606. In step 608, the sized data is then imported into Genotyper® using AppleScript™. In step 610, the Genotyper® is run to label the sized data with a genotype tag in the accepted convention (e.g., Marker identification and number of repeats), and in step 612 the Genotyper® formats the Genotyper® calls into a text table and exports the table for purposes of comparing the Genotyper® calls with allele calls obtained from another method.

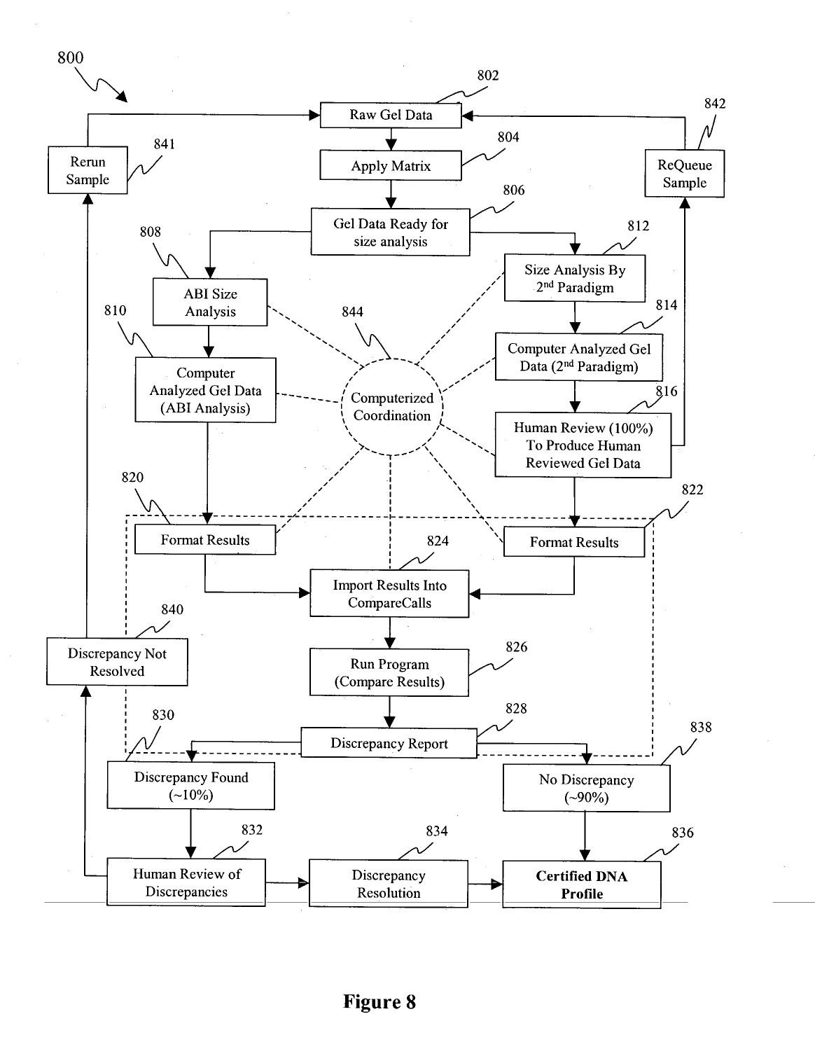

FIGS. 8-12 are flowcharts illustrating different variations in the STR profiling method described above and shown in FIG. 5. In FIG. 8, process 800 starts with step 802 with unprocessed gel image data (in the form of an electrical signal) ready for processing and analysis. A matrix algorithm is applied to the image data in step 804, after which the processed image data (in the form of an electrical signal) is ready for size analysis. Size analysis is conducted in two different paradigms. In the first, the ABI PRISM® Genotyper® Software and the GeneScan® Analysis Software are used in step 808 to provide a computer analyzed STR data set. Step 808 may include the steps of normalizing the electrical signal representing the unprocessed image data and converting the normalized first electrical signal to a first data set representing the identity of the STR markers and the number of repeats in the plurality of labeled DNA fragments. In addition, the same semi-processed data provided in step 804 is analyzed in step 812 using a second paradigm. The thus obtained second STR data set is then reviewed by a scientist in step 816. The data sets provided in steps 808 and 812 using two different analysis paradigms, respectively, are then formatted and subject to a computer-based comparison in steps 820, 822, 824, 826, and 828 to provide a discrepancy report or QA report. If there is no difference between the compared data sets, the STR profile is certified to be accurate. Otherwise, human review of the mismatching data is performed in an attempt to resolve the discrepancy. If the discrepancy cannot be resolved by human review, the sample is re-run to provide a second raw gel data which is subject to the same processing and analyzing procedures as described above.

Preferably, coordination means is provided to coordinate the two analysis processes and the comparison step as indicated in step 844 of the flowchart 800.

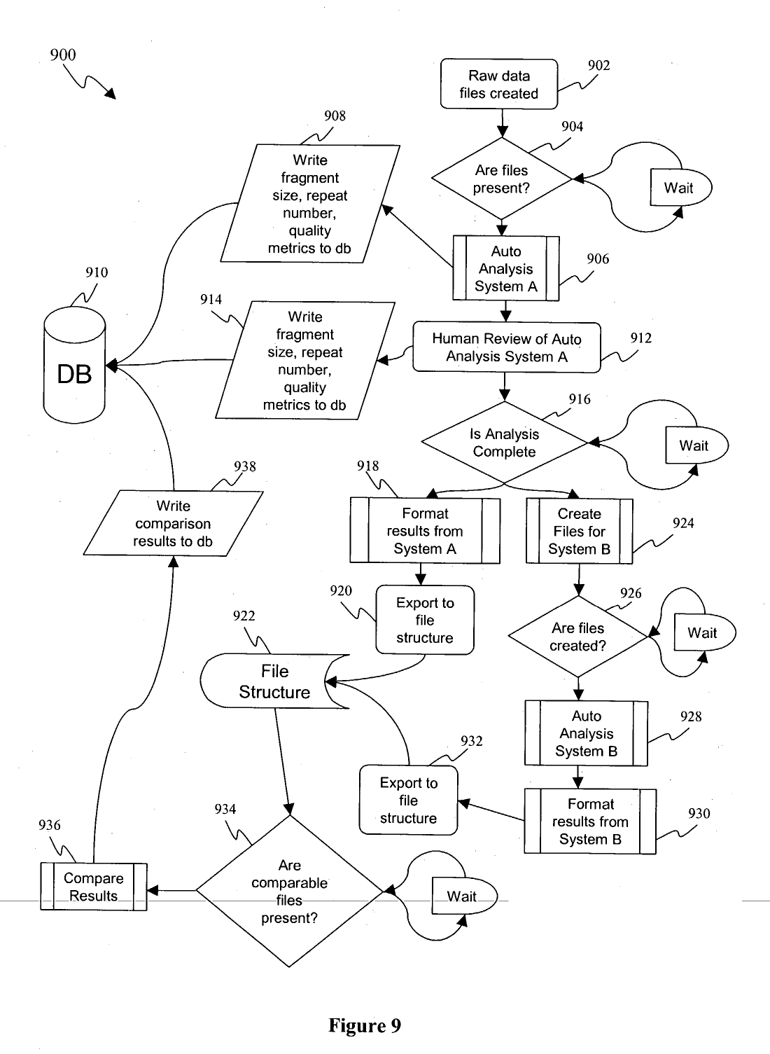

A similar and coordinated method is depicted in detail in FIG. 9. As shown in FIG. 9, flow process 900 begins with the creation of computer files of unprocessed image data (in the form of electrical signals) in step 902. The computer detects the presence of such files and proceeds to the analysis using System A in step 906. The result of this analysis (including DNA fragment sizes, number of STR, quality metrics, etc.) is then transferred to a database (steps 908 and 910). Optionally, human review is conducted in step 912 and the result is also transferred to the same or different database (steps 914 and 910). The computer-based coordination means then detects the completion of the analysis in system A (step 916) and initiate the steps to follow. Thus, the result from system A is formatted in step 918 and exported to a file structure (steps 920 and 922). In addition, files of unprocessed image data are also created for system B in step 924. Once the files are ready, System B is employed to converts the electrical signal to a second data set using a different algorithm that is utilized in System A (step 926 and 928). The result from System B is formatted in step 930 and exported to file structure 922. The computer-based coordination means then determines whether comparable files are present in the file structure (step 934), if the files are present, and initiates the computer-based comparison in step 936. The result of the comparison is written to the database in step 910.

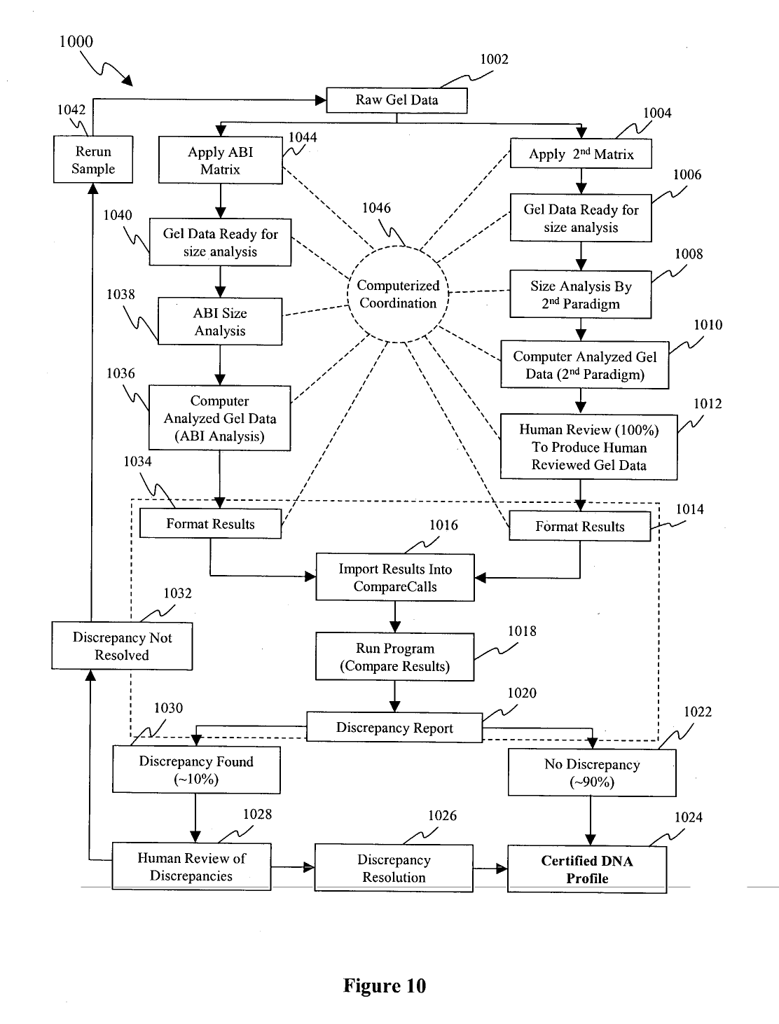

FIG. 10 shows a control flow process 1000 which is a variant of the process 800 provided above. In contrast to process 800 in which the same matrix is applied to the unprocessed image data to provide data ready for size analysis using two different paradigms process 1000 applies two different matrices to the raw gel data (steps 1004 and 1044), leading to two sets of data ready for size analysis using two different analysis paradigms.

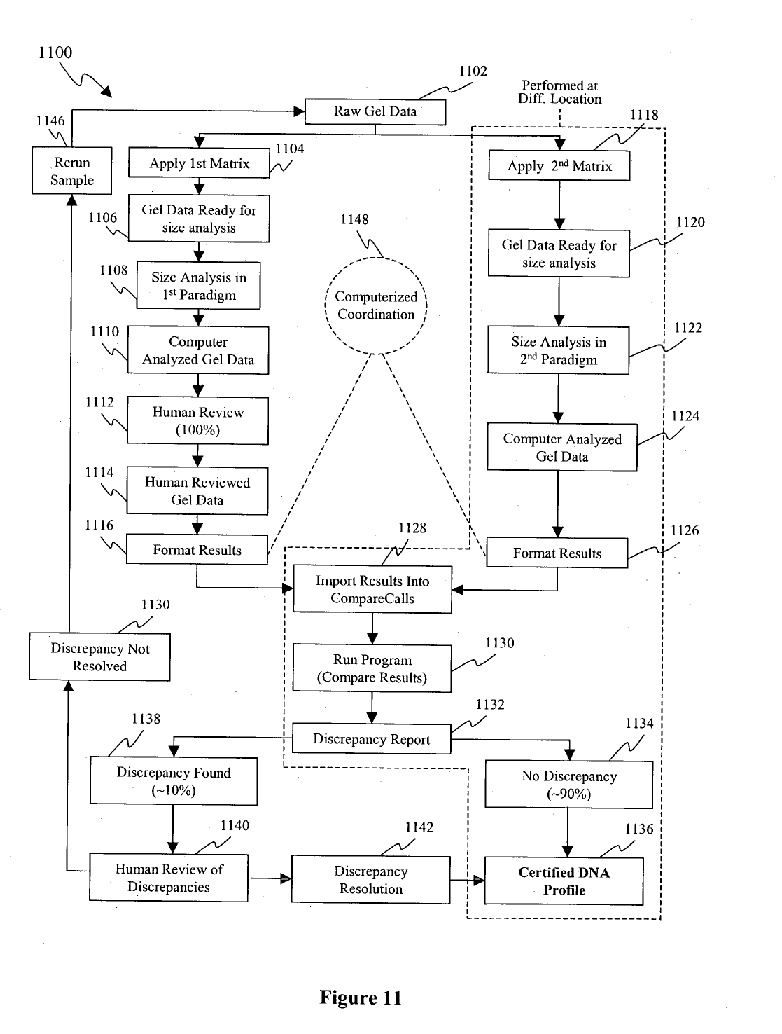

FIG. 11 depicts another flow process 1100, which is similar to process 1000. However, in process 1100, it is contemplated that raw gel data is generated and processed and analyzed (steps 1102, 1104, 1106, 1108, 1110, 1112, 1114, and 1116) in one physical location while quality control and data certification are conducted in another physical location away from the first one. That is, the raw gel data can be transmitted electronically through a communication infrastructure to the second location, in which data processing and analysis are performed using a second paradigm (steps 1118, 1120, 1122, 1124 and 1126) to generate a second set of formatted STR profile data which is compared to the first set of data from step 1116 in a computer-based comparison in steps 1128, 1130, and 1132.

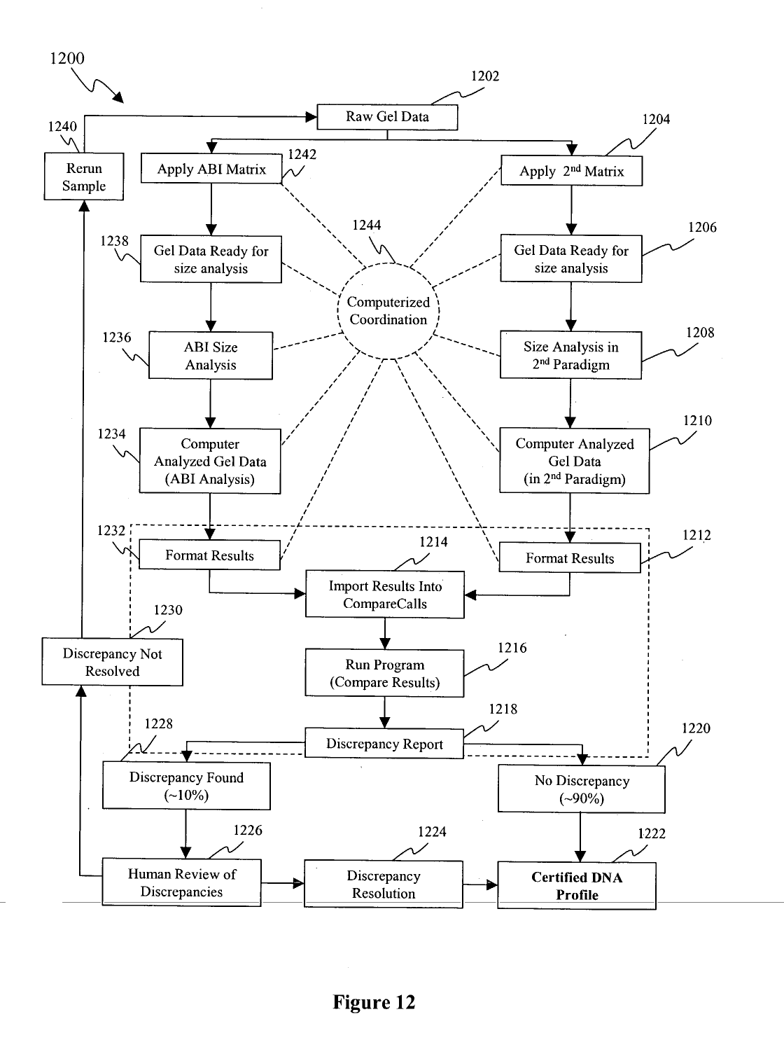

FIG. 12 illustrates yet another flow process 1200, which is a preferred embodiment of the method of the present invention. Process 1200 in FIG. 12 is similar to process 1000 in FIG. 10. However, the human review step (step 1012 in FIG. 10) for the computer processed and analyzed data before the computer-based comparison step is absent in process 1200. In other words, process 1200 is a completely automated process for data processing, analysis, and comparison to provide a certified DNA profile or a discrepancy report.

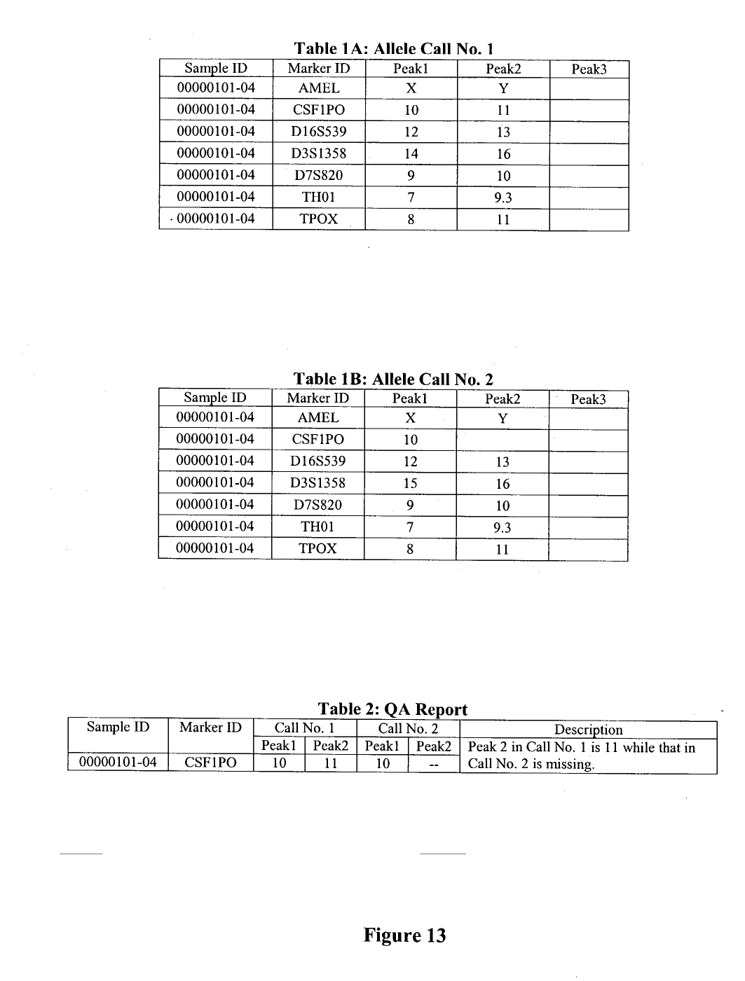

Table 1A in FIG. 13 illustrates an embodiment of a formatted table containing a set of STR profile data generated as a result of data analysis using a first paradigm. Similarly, Table 1B in FIG. 13 illustrates an embodiment of a formatted table containing a set of STR profile data generated as a result of data analysis using a second paradigm which is different from the first paradigm. Table 2 depicts an exemplary QA report generated in a computer-based comparison step comparing the data sets in Table 1A and Table 1B. As is apparent from FIG. 13, Table 2 identifies the mismatch in the two sets of data. The mismatch can be reviewed by a scientist to resolve the difference, or alternatively, the sample may be re-run to redo the analysis.

The method of the present invention is also applicable to microarray-based biochemical marker profiling, using nucleic acid or protein-based microarrays. As is known in the art, in nucleic acid-based microarrays or microchips, a large number of different oligonucleotide probes or cDNAs are attached or immobilized in an array on a solid support, e.g., a silicon chip or glass slide. Target nucleic acid sequences to be analyzed can be contacted with the immobilized oligonucleotide or cDNA probes on the microchip. See, e.g., U.S. Pat. No. 5,925,525 to Fodor et al;; Wilgenbus et al., J. Mol. Med., 77:761-786 (1999); Graber et al., Curr. Opin. Biotechnol., 9:14-18 (1998); Gingeras et al., Genome Res., 8:435-448 (1998); Drobyshev et al., Gene, 188:45-52 (1997); Shoemaker et al., Nat. Genet., 14:450-456 (1996); Kozal et al., Nat. Med. 2:753-759 (1996); DeRisi et al., Nat. Genet., 14:457-460 (1996); Chee et al., Nat. Genet., 14:610-614 (1996); Lockhart et al., Nat. Genet., 14:675-680 (1996); Lipshutz et al., Biotechniques, 19:442-447 (1995); Saiki et al., Proc. Natl. Acad. Sci. USA, 86:6230-6234 (1989).

Protein microarrays are also becoming increasingly important in both proteomics research and protein-based detection and diagnosis of diseases. Protein microarrays can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

Protein microarrays have also been made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Several protein microchips are commercially available. For example, the ProteinChip System from Ciphergen Biosystems Inc., Palo Alto, Calif. comprises metal chips having a treated surface, which interact with proteins. Basically, a metal chip surface is coated with a silicon dioxide film. The molecules of interest such as proteins and protein complexes can then be attached covalently to the chip surface via a silane coupling agent. Other commercially available protein chips include LabChip from Caliper Technology Corp. (Mountain View, Calif.), the Trinectin Proteome Chip from Phylos Inc. (Lexington, Mass.), the Triage protein chip from Biosite (San Diego, Calif.), the eTag Assay System from Aclara Biosciences (Hayward, Calif.), etc.

The microchip technologies combined with currently available computer-based data collection and analysis tools allow fast high throughput screening. However, the accuracy of the data derived from the screens is often compromised partly due to the large amount of data derived from the high throughput assays. In this respect, the present invention provides an ideal method for improving data quality without compromising the high efficiency.

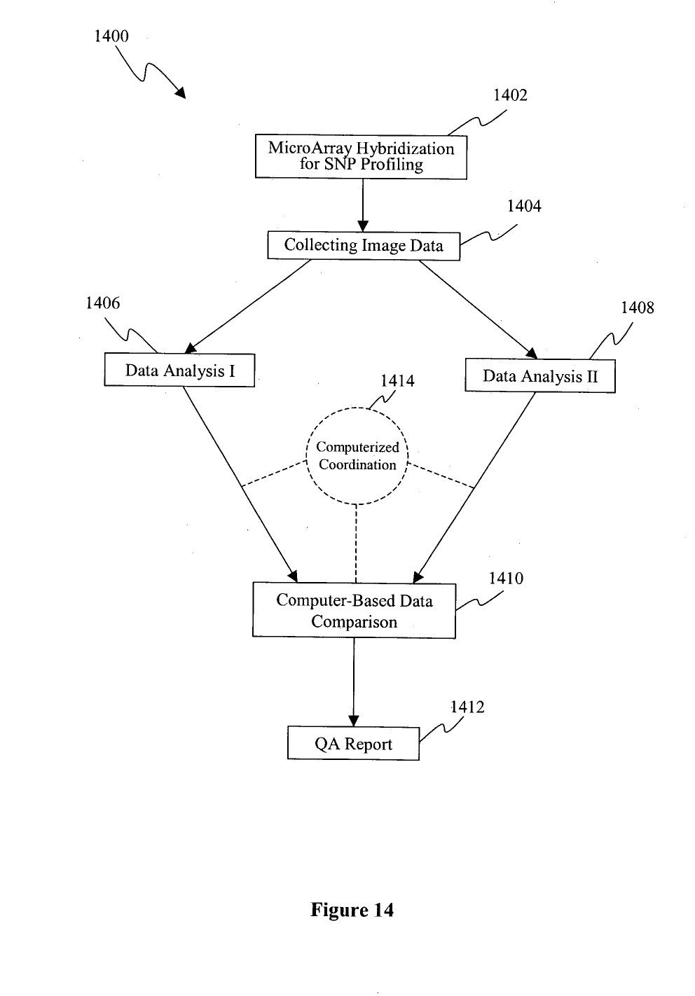

The method of the present invention is applicable to unprocessed image data obtained from any microarray screening assays, e.g., oligonucleotide-based SNP profiling, gene expression profiling, protein profiling, etc. FIG. 14 is a flowchart depicting flow process 1100, which applies the method of the present invention to a microarray-based SNP profiling using an oligonucleotide probe array. Such a microarray assay may optionally incorporate PCR-based assays, Sniper™-based rolling circle amplification (RCA) (commercially available from Amersham Pharmacia Biotech, and described in Clark & Pickering, Life Sci. News 6, 2000), and Invader®-based assays (commercially available from Third Wave Technologies, Inc., Madison, Wis.).

Referring to FIG. 14, samples are processed in step 1402 to prepare a hybridized microarray, from which unprocessed image data can be readily collected. Such a hybridized microarray can be prepared by hybridizing labeled amplified DNA fragments to a microarray of oligonucleotide probes. For example, specific regions of genomic DNA derived from a biological sample from an individual are amplified by, e.g., PCR and the fluorescence-labeled PCR products are hybridized to a microchip containing a plurality of addressable probes. In step 1404, fluorescence image data is collected (in the form of an electrical signal) from the microchip by, e.g., a GeneArray™ scanner (available from Affymetrix, Santa Clara, Calif.) or confocal microscope and converted to a computer-analyzable format. The unprocessed image data (in the form of an electrical signal) is then processed and analyzed using two different paradigms in steps 1406 and 1408, respectively. The two different paradigms utilize different computer algorithms. The thus obtained two different sets of data are then compared by computer means in step 1410, to generate a QA report in step 1412, either certifying the SNP profiling data or identifying the mismatches in the two data sets.

As a specific example, labeled samples can be applied to a microarray such as Affymetrix's GeneChip® HuSNP. The samples can then be allowed to hybridize to the microarray probes and an unprocessed image data of the microarray is prepared using a scanning device. Optionally, two different unprocessed image data are prepared using two different scanning devices or algorithms, respectively. For this purpose, scanners such as Affyrnetrix's GeneArray® Scanner, Agilent's DNA Microarray Scanner (e.g., Model G2565BA), and the like may be used. The unprocessed image data of the microarray (in the form of, e.g., an electrical signal) is then analyzed using two different paradigms. That is, the electrical signal is processed using two different methodologies and converted into two data sets, respectively, both corresponding to the identity of a plurality of loci and hybridization signal intensity at the plurality of loci of the microarray. The first may employ, e.g., the block indexing algorithm Modified k-nearest neighbors (MKNN) graph model and the spot indexing algorithm found in Jung and Cho, Bioinformatics, 18(Supp. 2):S141-S151 (2002). The spot intensity computation is then performed using the algorithm found in Kooperberg et al., J. Comput. Biol., 9(1):55-66 (2002). The second paradigm may utilize the block indexing algorithm k-nearest neighbors (KNN) graph model. The publicly available Matarray software algorithm may then be used to carry out spot detection and signal intensity calculations. See Wang et al., Nucleic Acids Research, 29(15) e75 (2001).

Preferably, the two data analysis processes using different paradigms or algorithms and the step of comparing the two data sets, are coordinated by a computer program means as described above. See, e.g., step 144 in FIG. 14.

A QA report can be generated as a result of the comparison of the two data sets. The QA report can indicate the matches and/or mismatches between the two data sets. The QA report can also track the quality of the two analysis processes which produce the two data sets. When no sufficient number of discrepancies (or mismatches) is found between the two data sets, the computer certifies the results. It is noted that the stringency of the requirements for certifying the array profile can vary. That is, the allowable number and/or degree of discrepancies between the two data sets for the computer to certify the results can be preset by users, as will be apparent to skilled artisans.

In accordance with another aspect, the present invention relates to systems for carrying out the method of the present invention. Generally, the present invention provides systems for analyzing image data to provide a certified biochemical profile of biological sample, which includes a data set having a plurality of data objects representing a plurality of molecular markers. The systems generally comprise a processor capable of effecting the following steps within the system: (1) providing, from an electrical signal representing an unprocessed image data and by a first process, a first data set corresponding to the plurality of molecular markers of the biological sample; (2) providing, from the electrical signal and by a second process, a second data set corresponding to the plurality of biological markers of the biological sample; and (3) comparing, by computer program means, the first and second data sets to determine the presence or absence of a discrepancy between the first and second data sets and to produce a QA report. The first and second processes for providing the first and second data sets typically include normalizing the electrical signal and converting the normalized electrical signal to a data set having a plurality of data objects representing a plurality of molecular markers. The first process is different from the second process. That is, the two processes employ different paradigms or algorithms. In preferred embodiments, the system further comprises another computer program means for coordinating the providing steps and the comparing step such that the comparing step is performed when the first data set and the second data set are both provided in comparable formats.

In one embodiment, a system for DNA fragments profiling (e.g., STR profiling, DNA sequencing, etc.) is provided. The system includes a sample analyzer for separating a plurality of labeled DNA fragments. The system may further include a data collector for producing an electrical signal representing unprocessed image data of the plurality of labeled DNA fragments. In addition, the system comprises a processor capable of effecting the following steps within the system: (1) providing, from the electrical signal and by a first process, a first data set corresponding to the number of short tandem repeats in each of the plurality of labeled DNA fragments, the first process comprising the steps of normalizing the electrical signal, and converting the normalized electrical signal to the first data set; (2) providing, from the electrical signal and by a second process, a second data set corresponding to the number of short tandem repeats in each of the plurality of labeled DNA fragments, the second process comprising the steps of normalizing the electrical signal and converting the normalized electrical signal to the second data set, wherein the first process is different from the second process; and (3) comparing the first and second data sets to determine the presence or absence of a discrepancy between the first and second data sets and to produce a QA report. Preferably, the processor in the system is also capable of effecting the coordination of the providing steps and the comparing step.

In another embodiment, the system for DNA profiling (e.g., STR profiling and the like) includes (1) a sample analyzer for separating a plurality of labeled DNA fragments; (2) a data collector for producing an electrical signal representing unprocessed image data of the plurality of labeled DNA fragments; (3) an interface module for receiving from the data collector the electrical signal; (4) a first computer program means for producing, from the electrical signal and by a first process, a first data set corresponding to the number of short tandem repeats in each of the plurality of labeled DNA fragments; (5) a second computer program means for producing, from the electrical signal and by a second process, a second data set corresponding to the number of short tandem repeats in each of the plurality of labeled DNA fragments, wherein the first and second computer program means are different from each other; and (6) a third computer program means for comparing the first and second data sets to determine the presence or absence of a discrepancy between the first and second data sets and to produce a QA report. Preferably, the system further comprises a fourth computer program means interfacing with the first, second and third computer program means to coordinate the progress of the data analysis and comparison processes.

In yet another embodiment, the system for DNA profiling (e.g., STR profiling) of a biological sample comprises: (1) a first interface module for receiving an electrical signal representing unprocessed image data of a plurality of labeled DNA fragments; (2) a first computer program means for producing, from the electrical signal and by a first process, a first data set corresponding to the number of short tandem repeats in each of the plurality of labeled DNA fragments; (3) a second interface module for receiving a second data set corresponding to the number of short tandem repeats in each of the plurality of labeled DNA fragments, the second data set being produced from the electrical signal by a second process, wherein the first and second processes are different from each other; and (4) a second computer program means for comparing the first and second data sets to determine the presence or absence of a discrepancy between the first and second data sets and to produce a QA report. Preferably, in this embodiment, the system further comprises a third computer program means which allows the comparison by the second computer program means to be initiated once both the first and second data sets are available in comparable formats. More preferably, the third computer program means functions to coordinate the first and second computer program means, and at least the second interface module to optimize the operation of the system. Specifically, the system is constructed such that it automatically monitors the progress of the process enabled by the first computer program means, detects the availability of the second data set at the second interface module, and controls the second computer program means to automatically compare the first and second data sets when available in comparable formats.

In yet another embodiment, the system of the present invention is suitable for profiling a microarray having proteins, nucleic acids, tissues or cells in a plurality of loci of the microarray. The system may optionally include a sample analyzer, e.g., a microarray with a plurality of addressable nucleic acid probes or antibodies. The system may also include a data collector for producing an electrical signal representing unprocessed image data from a microarray emitting detectable signals. The system also includes a processor capable of effecting the following steps within the system: (1) producing a first data set corresponding to the identity of the plurality of loci and image intensity at the plurality of loci, by a first process from the electrical signal, the first process comprising the steps of normalizing the electrical signal, and converting the electrical signal to the first data set; (2) producing, from the electrical signal and by a second process, a second data set corresponding to the identity of the plurality of loci and image intensity at the plurality of loci, the second process comprising converting the electrical signal to the second data set, wherein the second process is different from the first process; (3) comparing the first data set with the second data set to determine the presence or absence of a discrepancy between the first and second data sets; and optionally, (4) producing a QA report. Preferably, the system is capable of effecting the coordination of the producing steps and the comparing step.

In another embodiment, the system for profiling a microarray comprises a data collector for producing an electrical signal representing unprocessed image data of the microarray, and an interface module for receiving from the data collector the electrical signal. In addition, the system comprises a first computer program means for producing, from the electrical signal and by a first process, a first data set corresponding to the identity of the plurality of loci and image intensity at the plurality of loci, and a second computer program means for producing, from the electrical signal and by a second process, a second data set corresponding to corresponding to the identity of the plurality of loci and image intensity at the plurality of loci, wherein the first and second computer program means are different from each other. The system also includes a third computer program means for comparing the first and second data sets to determine the presence or absence of a discrepancy between the first and second data sets and to produce a QA report. Preferably, the system comprises a fourth computer program means interfacing with the first, second and third computer program means to coordinate the progress thereof. The coordination program means may allow the comparison by the third computer program means to be initiated once both the first and second data sets are available in comparable formats. More preferably, the fourth computer program means functions to coordinate the first, second and third computer program means to optimize the operation of the system. Most preferably, the system is constructed such that the fourth computer program means (coordination means) automatically monitors the progress of the process enabled by the first and second computer program means, detects the availability of the first and second data sets, and controls the third computer program means to automatically compare the first and second data sets when available in comparable formats.

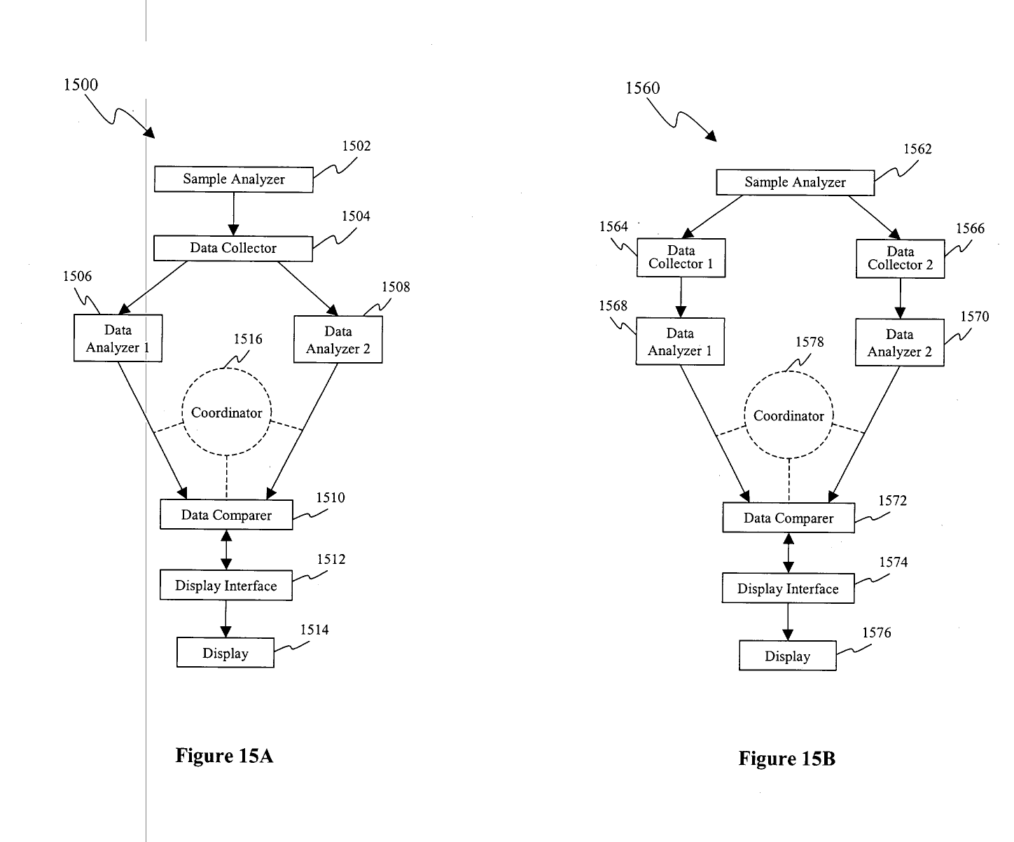

Referring to FIG. 15A, the system 1500 includes a sample analyzer 1502, which processes samples such that raw image data can be readily obtained from the sample analyzer. Examples of sample analyzers suitable for STR profiling include, e.g., ABI PRISM® 310 Genetic Analyzer, the ABI PRISM® 3700 DNA Analyzer and the ABI PRISM® 377 DNA Sequencer (all commercially available from Applied Biosystems, Inc., Foster City, Calif.), the MegaBace DNA analysis instruments from Amersham BioSciences, Piscataway, N.J., and the Hitachi FMBIO® II from Hitachi Inc. Examples of suitable sample analyzer suitable for microarray-based SNP profiling include, but are not limited to GeneChip® Probe Arrays and GeneChip® Instrument Systems (both available from Affymetrix Inc. (Santa Clara, Calif.), and the like. In addition, system 1500 includes a data collector 1504, which acquires image data from the sample analyzer. The image data acquired is normally in the form of an electrical signal. A data collector for DNA profiling (e.g., STR profiling) can be a tracking device such as a laser scanning monitor in conjunction with a data collection software. Examples of data collectors for microarrays include various scanning equipments such as Agilent DNA Microarray Scanner, GenePix® 400B Microarray Scanner from Axon Instruments, Inc., the array WoRx^{e} system from Applied Precision Inc., GeneArray® Scanner from Affymetrix, and the like.

In addition, system 1500 also includes two data analyzers 1506 and 1508 for processing and analyzing the raw image data collected by data collector 1504. Data analyzers 1506 and 1508 utilize different data processing and analysis paradigms or algorithms, which can be embodied in computer software or computer-readable instruction codes. However, Data analyzers 1506 and 1508 may share the same hardware or processor for running the software or computer-readable codes. Commercially available data analyzers suitable for size-based analysis (e.g., STR profiling) include, e.g., ABI PRISM® Genotyper® Software (Applied Biosystems, Inc., Foster City, Calif.), Hitachi FMBIO® Analysis Software (available from Promega Inc., Madison, Wis.) and TrueAllele™ (commercially available from Cytogenetics, Co., Pittsburgh, Pa.). Examples of commercially available software for analyzing microarray image data include, e.g., ImageMaster Array from Amersham BioScience, Agilent Feature Extraction Software, GenePix from Axon Instruments, Inc., etc.

In addition, system 1500 also includes a data comparer 1510, which can be embodied in computer software or hardware for comparing the data sets generated by data analyzers 1506 and 1508. Optionally, system 1500 further includes a display interface 1512 and display 1514 for displaying the result (certification or QA report showing accordance or discordance) of the computer-based comparison result generated by data comparer 1510. Preferably, system 1500 further includes a coordinator embodied in computer software or hardware. The coordinator can detect data sets generated by the data analyzers 1506 and 1508, and controls the initiation of the data comparer 1510 in comparing the data sets. More preferably, the coordinator interfaces with the two data analyzers and data comparer to coordinate and/or optimize the operation of the different components of the system.

It is noted that in system 1500 of the present invention, the data comparer and coordinator can be embodied solely in software or computer-readable codes, which are run by a computer processor in sample analyzer 1502 or data collector 1504, or data analyzer 1506 or 1508. Alternatively, data comparer 1510 and coordinator 1516 may comprise software or computer-readable codes and a processor for executing the software or computer-readable codes.

FIG. 15B illustrates system 1560, which is a variant of system 1500. System 1560 differs from system 1500 in that two data collectors (1564 and 1566) are included in system 1560. The two data collectors each produces an electrical signal from the same sample analyzer corresponding to the image on the sample analyzer.

The method of the present invention can also be embodied in computer program products and used in the systems described above or other computer- or internet-based systems. Accordingly, another aspect of the present invention relates to a computer program product comprising a computer-usable medium having computer-readable program code or instructions embodied thereon for enabling a processor to carry out the method of the present invention.

In one embodiment, a computer program product is provided for providing a certified biochemical profile of a biological sample having a data set including a plurality of data objects representing a plurality of molecular markers. The computer program product comprising a computer-usable medium having computer-readable program code embodied thereon for effecting a computing system to receive a first data set corresponding to the plurality of molecular markers of the biological sample, wherein the first data set is converted from an electrical signal corresponding to an unprocessed image data by a first process, the first process comprising the steps of normalizing the electrical signal, and converting the normalized electrical signal to the first data set. The computer-readable program code may also effect the computing system to receive a second data set corresponding to the plurality of molecular markers of the biological sample, wherein the second data set is converted from an electrical signal corresponding to an unprocessed image data by a second process, the second process comprising the steps of normalizing the electrical signal and converting the normalized electrical signal to the first data set, wherein the first process is different from the second process. In addition, the computer-readable program code may also enable the computing system to compare the first and second data sets to determine the presence or absence of a discrepancy between the first and second data sets and to produce a QA report. Preferably, the computer-readable program code also enables the computing system to coordinate the first and second processes such that the comparing step is automatically performed when the first data set and the second data set are both provided in comparable formats.

In another embodiment, the computer program product comprises a computer-usable medium having computer-readable program code embodied thereon capable of enabling a computing system to provide, from an electrical signal corresponding to an unprocessed image data and by a first process, a first data set corresponding to the plurality of molecular markers of the biological sample, the first process comprising the steps of normalizing the electrical signal, and converting the normalized electrical signal to the first data set. The computer-readable program code may also enable the computing system to provide, from the electrical signal and by a second process, a second data set corresponding to the plurality of biological markers of the biological sample, the second process comprising the steps of normalizing the electrical signal and converting the normalized electrical signal to the second data set, wherein the first process is different from the second process. For example, in STR profiling, each of the analysis paradigms may include (1) means for trace processing including baseline subtraction, spectral separation and peak smoothing; (2) fragment sizing to calculate the length of the DNA fragments and identifies the peaks in the sample data by comparison with size standards; and (3) allele calling on the amplified STR fragments. The two different paradigms should employ one or more different algorithms.

In addition, the computer-readable program code may also effect a step of comparing the first and second data sets to determine the presence or absence of a discrepancy between the first and second data sets and to produce a QA report. An example of algorithm for the comparison step is illustrated in FIG. 2 described above.

Preferably, the computer-readable program code can also enable the computing system to coordinate the providing steps and the comparing step such that the comparing step is automatically performed when the first data set and the second data set are both provided in comparable formats. More preferably, the computer-readable program code can enable the coordination of the progress of the steps of providing steps and the comparing step. It may also allow the comparison of the data sets to be initiated when both the first and second data sets are available in comparable formats. Most preferably, the computer-readable program code enables automatically monitoring the progress of the processes of providing the first and second data sets, detecting the availability of the first and second data sets, and initiating the comparing step. Examples of coordination algorithms are illustrated in FIGS. 3 and 4 described above.

It will be understood that each block or step of the flowcharts illustration and combinations of blocks in the flowcharts can be implemented by computer program instructions. These computer program instructions may be loaded onto a computer or other programmable apparatus to produce a machine, such that the instructions which execute on the computer or other programmable apparatus create means for implementing the functions specified in the flowcharts or step(s). These computer program instructions may also be stored in a computer-readable memory or medium that can direct a computer or other programmable apparatus to function in a particular manner, such that the instructions stored in the computer-readable memory or medium produce an article of manufacture including instruction means which implement the function specified in the flowcharts or step(s). The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions specified in the flowcharts or step(s).

Accordingly, the flowcharts support combinations of means for performing the specified functions, combinations of steps for performing the specified functions and program instruction means for performing the specified functions. It will also be understood that each step of the flowcharts and combinations of steps in flowcharts can be implemented by special purpose hardware-based computer systems, which perform the specified functions or steps, or combinations of special purpose hardware and computer instructions.

Accordingly, in accordance with yet another aspect, a computer system is provided for executing the instructions from the computer program products of the present invention. An embodiment of the system is shown in FIG. 16. Referring now to FIG. 16, system 1600 includes one or more processors, such as processors 1602 and 1604. The processors can be used to execute software or computer-readable instruction codes implementing the method of the present invention described above. It must be understood that the processor may consist of any number of devices. The processor may be a data processing device, such as a microprocessor or microcontroller or a central processing unit. The processor could be another logic device such as a DMA (Direct Memory Access) processor, an integrated communication processor device, a custom VLSI (Very Large Scale Integration) device or an ASIC (Application Specific Integrated Circuit) device. In addition, the processor can be any other type of analog or digital circuitry that is designed to perform the processing functions described herein.

Computer system 1600 also includes a main memory 1618, preferably random access memory (RAM). A second memory 1606 may also be included. The secondary memory 1606 may include, e.g., a remove storage drive 1608, which can be in various forms, including but not limited to, a magnetic tape drive, a floppy disk drive, a VCD drive, a DVD drive, an optical disk drive, etc. The removable storage drive 1608 may be compatible with a removable storage unit 1610 such that it can read from and/or write to the removable storage unit 1610. Removable storage units typically include a computer usable storage medium having stored therein computer-readable program codes or instructions and/or computer readable data. Example of removable storage units are well known in the art, including, but not limited to, floppy disks, magnetic tapes, optical disks, and the like.

Preferably, secondary memory 1606 also includes a hard drive 1612, which can be used to store computer readable program codes or instructions, and/or computer readable data.

In addition, as shown in FIG. 16, secondary memory 1606 may further include an interface 1614 and a removable storage unit 1616 that is compatible with interface 1614 such that software, computer readable codes or instructions can be transferred from the removable storage unit 1616 into computer system 1600. Examples of interface-removable storage unit pairs include, e.g., removable memory chips (e.g., EPROMs or PROMs) and sockets associated therewith, program cartridges and cartridge interface and the like.

As shown in FIG. 16, in computer system 1600, processors 1602 and 1604 as well as the main and secondary memories 1618 and 1606 are all operably linked together through communication infrastructure 1620, which may be a communications bus, system board, cross-bar, etc.). Through the communication infrastructure 1620, computer program codes or instructions or computer readable data can be transferred and exchanged.

Additionally, as discussed above especially in the context of FIG. 11, it may also be desirable to exchange data with other devices or systems located distant from the computer system 1600. For this purpose, an internet node or an intranet node including an interface module (also called communications interface) 1626 may be included in computer system 1600 such that computer-readable data (e.g., texts, tables, descriptions, photos, diagrams, etc. in the form of electronic, electromagnetic, optical or other signals), and software or other computer-readable codes or instructions may be transferred back and forth between external device 1630 and computer system 1600. Preferably, a communications path 1628 compatible with the communications interface 1626 is included. As will be apparent to skilled artisans, modems, communication ports, network cards such as Ethernet cards, and newly developed devices for accessing intranet or internet can all be used as the communication interface 1626.

In addition, computer system 1600 can also include a display interface 1622. Through display interface 1622, results of data analysis, e.g., in the forms of graphics, table, text, and the like from communication infrastructure 1620 may be displayed on display unit 1624.

In accordance with the present invention, the computer system implements a computer program or computer-readable codes or instructions to execute the data processing, analysis, comparison and coordination described above in various embodiments of the present invention. As such the computer readable program codes or instructions (i.e., computer instructions means for enabling processor 1602 and/or 1604 to perform the processing, analysis, comparison and/or coordination) may be included in the computer system such as system 1600 directly or through a computer program product as described above. Examples of such computer program products in system 1600 include, e.g., removable storage units 1610 and 1616, a hard disk (not shown) in hard disk drive 1612 and a carrier wave (not shown) which delivers software or computer readable codes or instructions to system 1600 through communication interface 1626 and communication path 1628.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for providing a certified biochemical profile of a biological sample, said biochemical profile having a data set including a plurality of data objects representing a plurality of molecular markers, said method comprising:
providing, from a first electrical signal corresponding to a first unprocessed image data and by a first process, a first data set representing the plurality of molecular markers of the biological sample;
providing, from a second electrical signal corresponding to a second unprocessed image data and by a second process, a second data set representing said plurality of biological markers of the biological sample, wherein said first process is different from said second process; and
comparing, by a computer-readable program code, said first and second data sets to detect discrepancies between said first and second data sets.

2. The method of claim 1, wherein the steps of said method are coordinated by a second computer-readable program code such that said comparing step is performed when said first and second data sets are both provided in comparable format.

3. The method of claim 1, wherein said first unprocessed image data is said second unprocessed image data.

4. The method of claim 1, wherein said first unprocessed image data is different from said second unprocessed image data, and said first and second unprocessed image data are obtained from a single image.

5. The method of claim 1, wherein said first process comprises the steps of:
normalizing said first electrical signal; and
converting the normalized first electrical signal to said first data set.

6. A method for providing a QA report on a biochemical profile of a biological sample, said biochemical profile having a data set including a plurality of data objects representing a plurality of molecular markers, said method comprising:
providing, from a first electrical signal corresponding to a first unprocessed image data and by a first process, a first data set corresponding to the plurality of molecular markers of the biological sample, said first process comprising the steps of normalizing said first electrical signal, and converting the normalized first electrical signal to said first data set;
providing, from a second electrical signal corresponding to a second unprocessed image data and by a second process, a second data set corresponding to said plurality of biological markers of the biological sample, said second process comprising the steps of normalizing said second electrical signal and converting the normalized second electrical signal to said second data set, wherein said first process is different from said second process; and
comparing, by a first computer-readable program code, said first and second data sets to produce a QA report, wherein said providing steps and said comparing step are coordinated by a second computer-readable program code such that said comparing step is performed when said first data set and said second data set are both provided in a comparable format.

7. A method for providing a biochemical profile of a biological sample, said biochemical profile having a data set including a plurality of data objects representing a plurality of molecular markers, said method comprising:
providing, from an electrical signal corresponding to an unprocessed image data and by a first process, a first data set corresponding to the plurality of molecular markers of the biological sample, said first process comprising the steps of normalizing said electrical signal, and converting the normalized electrical signal to said first data set;
providing, from said electrical signal and by a second process, a second data set corresponding to said plurality of biological markers of the biological sample, said second process comprising the steps of normalizing said electrical signal and converting the normalized electrical signal to said second data set, wherein said first process is different from said second process; and
comparing, by a first computer-readable program code, said first and second data sets to determine the presence or absence of a discrepancy between said first and second data sets and to produce a QA report, wherein said providing steps and said comparing step are coordinated by a second computer-readable program code such that said comparing step is performed when said first data set and said second data set are both provided in a comparable format.

8. A method for providing a certified DNA profile of an individual, comprising:
producing a plurality of DNA fragments characterizing an individual;
analyzing said plurality of DNA fragments using a first analysis means to provide a first data set corresponding to said plurality of DNA fragments;
analyzing said plurality of DNA fragments using a second analysis means that is different from said first analysis means to provide a second data set corresponding to said plurality of DNA fragments; and
comparing, by computer program means, said first data set with said second data set to determine the presence or absence of a discrepancy between said first and second data sets, wherein said computer program means certifies said first and second data set in the absence of discrepancy.

9. The method of claim 8, wherein said analyzing steps and said comparing step are coordinated by computer program means such that said comparing step is performed when said first and second data sets are both provided in a comparable format.

10. A method for providing a DNA STR profile of an individual, comprising:
producing a plurality of labeled DNA fragments having detectable labels and corresponding to said DNA STR profile of said individual;
producing an electrical signal representing unprocessed image data of said labeled DNA fragments;
providing a first data set representative of the number of short tandem repeats in each of said plurality of labeled DNA fragments, by a first process from said electrical signal, said first process comprising the steps of normalizing said electrical signal, and converting said electrical signal to said first data set;
providing a second data set representative of the number of short tandem repeats in each of said plurality of labeled DNA fragments, by a second process from said electrical signal, said second process comprising converting said electrical signal to said second data set, wherein said second process is different from said first process; and
comparing, by a computer program code, said first data set with said second data set to determine the presence or absence of a discrepancy between said first and second data sets and to provide a QA report, wherein a certified STR profile is provided comprising said first or second data set in the absence of the discrepancy.

11. The method of claim 10, wherein said providing steps and said comparing step are coordinated by a second computer-readable program code such that said comparing step is performed when said first plurality of data objects and said second plurality of data objects are both provided in a comparable format.

12. The method of claim 10, wherein said step of producing said electrical signal representing unprocessed image data of said labeled DNA fragments comprises separating said plurality of labeled DNA fragments and converting the labels into said electrical signal.

13. A method for analyzing an image data obtained from a microarray having proteins, nucleic acids, tissues or cells in a plurality of loci of the microarray, said method comprising:
providing an electrical signal representing unprocessed image data from said microarray;
producing a first data set representative of the identity of said plurality of loci and image intensity at said plurality of loci, by a first process from said electrical signal, said first process comprising the steps of normalizing said electrical signal, and converting said electrical signal to said first data set;
producing, from said electrical signal and by a second process, a second data set representative of the identity of said plurality of loci and image intensity at said plurality of loci, said second process comprising converting said electrical signal to said second data set, wherein said second process is different from said first process; and
comparing, by a first computer-readable program code, said first data set with said second data set to determine the presence or absence of a discrepancy between said first and second data sets, wherein said producing steps and comparing step are coordinated by a second computer-readable program code such that said comparing step is performed when said first data set and said second data set are both provided in a comparable format.

14. The method of claim 13, wherein said microarray is a nucleic acid array having hybridized thereto labeled DNA or RNA molecules.

15. The method of claim 13, wherein said microarray is a protein array having labeled proteins attached thereto.

16. The method of claim 13, wherein said microarray is a cell array.

17. A system for analyzing image data to provide a certified biochemical profile of a biological sample, said biochemical profile having a data set including a plurality of data objects representing a plurality of molecular markers, wherein said system comprises a processor capable of effecting the following steps within the system:
providing, from an electrical signal corresponding to an unprocessed image data and by a first process, a first data set corresponding to the plurality of molecular markers of the biological sample, said first process comprising the steps of normalizing said electrical signal and converting the normalized electrical signal to said first data set;
providing, from said electrical signal and by a second process, a second data set corresponding to said plurality of biological markers of the biological sample, said second process comprising the steps of normalizing said electrical signal and converting the normalized electrical signal to said second data set, wherein said first process is different from said second process; and
comparing, by a first computer-readable program code, said first and second data sets to determine the presence or absence of a discrepancy between said first and second data sets and to produce a QA report.

18. The method of claim 17, wherein said providing steps and said comparing step are coordinated by a second computer-readable program code such that said comparing step is performed when said first data set and said second data set are both provided in a comparable format.

19. A system for DNA STR profiling, comprising:
a sample analyzer for separating a plurality of labeled DNA fragments;
a data collector for producing an electrical signal representing unprocessed image data of said plurality of labeled DNA fragments wherein said system comprises a processor capable of effecting the following steps within the system:
providing, from said electrical signal and by a first process, a first data set corresponding to the number of short tandem repeats in each of said plurality of labeled DNA fragments, said first process comprising the steps of normalizing said electrical signal, and converting the normalized electrical signal to said first data set;
providing, from said electrical signal and by a second process, a second data set corresponding to the number of short tandem repeats in each of said plurality of labeled DNA fragments, said second process comprising the steps of normalizing said electrical signal and converting the normalized electrical signal to said second data set, wherein said first process is different from said second process; and
comparing said first and second data sets to determine the presence or absence of a discrepancy between said first and second data sets and to produce a QA report.

20. The system of claim 19, wherein said processor is capable of effecting the coordination of said providing steps and said comparing step.

21. A system for DNA STR profiling, comprising:
a sample analyzer for separating a plurality of labeled DNA fragments;
a data collector for producing an electrical signal representing unprocessed image data of said plurality of labeled DNA fragments;
an interface module for receiving from said data collector said electrical signal;
a first computer program means for producing, from said electrical signal and by a first process, a first data set corresponding to the number of short tandem repeats in each of said plurality of labeled DNA fragments;
a second computer program means for producing, from said electrical signal and by a second process, a second data set corresponding to the number of short tandem repeats in each of said plurality of labeled DNA fragments, wherein said first and second computer program means are different from each other; and
a third computer program means for comparing said first and second data sets to determine the presence or absence of a discrepancy between said first and second data sets and to produce a QA report.

22. The system of claim 21, further comprising a fourth computer program means interfacing with said first, second and third computer program means to coordinate their progress.

23. A system for DNA STR profiling of a biological sample, comprising:
a first interface module for receiving an electrical signal representing unprocessed image data of a plurality of labeled DNA fragments;
a first computer program means for producing, from said electrical signal and by a first process a first data set corresponding to the number of short tandem repeats in each of said plurality of labeled DNA fragments;
a second interface module for receiving a second data set corresponding to the number of short tandem repeats in each of said plurality of labeled DNA fragments, said second data set being produced from said electrical signal by a second process, wherein said first and second processes are different from each other; and
a second computer program means for comparing said first and second data sets to determine the presence or absence of a discrepancy between said first and second data sets and to produce a QA report.

24. A system for profiling a microarray having proteins, nucleic acids, tissues or cells in a plurality of loci of the microarray, said system comprising:
a data collector for producing an electrical signal representing unprocessed image data from said microarray, wherein said system comprises a processor capable of effecting the following steps within the system:
producing a first data set corresponding to the identity of said plurality of loci and image intensity at said plurality of loci, by a first process from said electrical signal, said first process comprising the steps of normalizing said electrical signal, and converting said electrical signal to said first data set;
producing, from said electrical signal and by a second process, a second data set corresponding to the identity of said plurality of loci and image intensity at said plurality of loci, said second process comprising converting said electrical signal to said second data set, wherein said second process is different from said first process;
comparing said first data set with said second data set to determine the presence or absence of a discrepancy between said first and second data sets; and
producing a QA report.

25. The system of claim 24, wherein said processor is capable of effecting the coordination of said producing steps and said comparing step.

26. A system for profiling a microarray having proteins, nucleic acids, tissues or cells in a plurality of loci of the microarray, said system comprising:
a data collector for producing an electrical signal representing unprocessed image data of said microarray;
an interface module for receiving from said data collector said electrical signal;
a first computer program means for producing, from said electrical signal and by a first process, a first data set corresponding to the identity of said plurality of loci and image intensity at said plurality of loci;
a second computer program means for producing, from said electrical signal and by a second process, a second data set corresponding to corresponding to the identity of said plurality of loci and image intensity at said plurality of loci, wherein said first and second computer program means are different from each other; and
a third computer program means for comparing said first and second data sets to determine the presence or absence of a discrepancy between said first and second data sets and to produce a QA report.

27. The system of claim 26, further comprising a fourth computer program means interfacing with said first, second and third computer program means to coordinate the progress thereof.

28. A system for profiling a microarray having proteins, nucleic acids, tissues or cells in a plurality of loci of the microarray, said system comprising:
a first interface module for receiving an electrical signal representing unprocessed image data of said microarray;
a first computer program means for producing, from said electrical signal and by a first process, a first data set corresponding to the identity of said plurality of loci and image intensity at said plurality of loci;
a second interface module for receiving a second data set corresponding to the identity of said plurality of loci and image intensity at said plurality of loci, said second data set being produced from said electrical signal by a second process, wherein said first and-second processes are different from each other;
a second computer program means for comparing said first and second data sets to determine the presence or absence of a discrepancy between said first and second data sets and to produce a QA report.

29. A computer program product for providing a certified biochemical profile of a biological sample, said biochemical profile having a data set including a plurality of data objects representing a plurality of molecular markers, said computer program product comprising a computer-usable medium having computer-readable program code embodied thereon for effecting the following steps within a computing system:
receiving a first data set corresponding to the plurality of molecular markers of the biological sample, wherein said first data set is converted from an electrical signal corresponding to an unprocessed image data by a first process, said first process comprising the steps of normalizing said electrical signal, and converting the normalized electrical signal to said first data set;
receiving a second data set corresponding to the plurality of molecular markers of the biological sample, wherein said second data set is converted from an electrical signal corresponding to an unprocessed image data by a second process, said second process comprising the steps of normalizing said electrical signal and converting the normalized electrical signal to said first data set, wherein said first process is different from said second process;
comparing said first and second data sets to determine the presence or absence of a discrepancy between said first and second data sets and to produce a QA report; and
coordinating said first and second processes such that said comparing step is performed when said first data set and said second data set are both provided in a comparable format.

30. The computer program product of claim 29, wherein said biochemical profile is a DNA STR profile.

31. The computer program product of claim 29, wherein said biochemical profile is a gene expression profile obtained from a nucleic acid microarray.

32. The computer program product of claim 29, wherein said biochemical profile is a genotype profile obtained from a nucleic acid microarray.

33. The computer program product of claim 29, wherein said biochemical profile is obtained from a protein microarray.

34. A computer program product for providing a certified biochemical profile of a biological sample, said biochemical profile having a data set including a plurality of data objects representing a plurality of molecular markers, said computer program product comprising a computer-usable medium having computer-readable program code embodied thereon for effecting the following steps within a computing system:
providing, from an electrical signal corresponding to an unprocessed image data and by a first process, a first data set corresponding to the plurality of molecular markers of the biological sample, said first process comprising the steps of normalizing said electrical signal, and converting the normalized electrical signal to said first data set;
providing, from said electrical signal and by a second process, a second data set corresponding to said plurality of biological markers of the biological sample, said second process comprising the steps of normalizing said electrical signal and converting the normalized electrical signal to said second data set, wherein said first process is different from said second process;
comparing said first and second data sets to determine the presence or absence of a discrepancy between said first and second data sets and to produce a QA report; and
coordinating said providing steps and said comparing step such that said comparing step is performed when said first data set and said second data set are both provided in a comparable format.
